(19) United States
(12) Patent Application Publication
(10) Pub. No.: US 2005/0112128 A1
(43) Pub. Date: May 26, 2005

(54) Inhibition of protein kinase c-mu (PKD) as a treatment for cardiac hypertrophy and heart failure

(75) Inventors: Timothy A. McKinsey, Broomfield, CO (US); Eric Olson, Dallas, TX (US); Rick B. Vega, Warwick, RI (US)

Correspondence Address: FULBRIGHT & JAWORSKI L.L.P., SUITE 2400, 600 CONGRESS AVENUE, AUSTIN, TX, 78701-3271, US

(73) Assignee: Myogen, Inc. and Board of Regents, The University of Texas System

(21) Appl. No.: 10/848,820

(22) Filed: May 19, 2004

Related U.S. Application Data
(60) Provisional application No. 60/472,298, filed on May 21, 2003.

(51) Int. Cl. A61K 48/00; A61K 38/10; A61K 38/08; A61K 31/553; A61K 31/704; A61K 31/407

(52) U.S. Cl. 424/146.1; 514/211.08; 514/16; 514/17; 514/44; 514/410; 514/733; 514/651; 514/355; 514/423; 514/223.5; 514/27; 514/456

(57) ABSTRACT

The present invention provides for methods of treating and preventing cardiac hypertrophy and heart failure. MEF-2 and Class II HDACs have been shown to have a major role in cardiac hypertrophy and heart disease, and inhibition of class II HDAC's has been shown to have a beneficial, anti-hypertrophic effect. The present invention provides the link between MEF-2 and class II HDAC's, a kinase known as PKD. The present invention further demonstrates that inhibitors of PKD inhibit cardiac hypertrophy and heart disease by inhibiting, in part, the fetal cardiac gene expression and cellular reorganization that occurs when MEF-2 dependent transcription is inhibited.

The present invention claims priority to U.S. Provisional Ser. No. 60/472,298, filed May 21, 2003, the entire contents of which are hereby incorporated by reference.

The United States government owns rights in the application by virtue of funding under Grant No. P01 HL61544 from the National Institutes of Health.

### BACKGROUND OF THE INVENTION

1. Field of the Invention

The present invention relates generally to the fields of developmental biology and molecular biology. More particularly, it concerns gene regulation and cellular physiology in cardiomyocytes. Specifically, the invention relates to the use inhibitors of Protein Kinase C-μ (PKD) to block phosphorylation of histone deacetylases. It also relates to the use of PKD inhibitors to treat cardiac hypertrophy and heart failure.

2. Description of Related Art

Cardiac hypertrophy in response to an increased workload imposed on the heart is a fundamental adaptive mechanism. It is a specialized process reflecting a quantitative increase in cell size and mass (rather than cell number) as the result of any, or a combination of, neural, endocrine or mechanical stimuli. Hypertension, another factor involved in cardiac hypertrophy, is a frequent precursor of congestive heart failure. When heart failure occurs, the left ventricle usually is hypertrophied and dilated and indices of systolic function, such as ejection fraction, are reduced. Clearly, the cardiac hypertrophic response is a complex syndrome and the elucidation of the pathways leading to cardiac hypertrophy will be beneficial in the treatment of heart disease resulting from various stimuli.

A family of transcription factors, the myocyte enhancer factor-2 family (MEF2), is involved in cardiac hypertrophy. For example, a variety of stimuli can elevate intracellular calcium, resulting in a cascade of intracellular signaling systems or pathways, including calcineurin, CAM kinases, PKC and MAP kinases. All of these signals activate MEF2 and result in cardiac hypertrophy. However, it is still not completely understood how the various signal systems exert their effects on MEF2 and modulate its hypertrophic signaling. It is known that certain histone deacetylase proteins (HDAC's) are involved in modulating MEF2 activity.

Eleven different HDACs have been cloned from vertebrate organisms. All share homology in the catalytic region. Histone acetylases and deacetylases play a major role in the control of gene expression. The balance between activities of histone acetylases, usually called acetyl transferases (HATs), and deacetylases (HDACs) determines the level of histone acetylation. Consequently, acetylated histones cause relaxation of chromatin and activation of gene transcription, whereas deacetylated chromatin is generally transcriptionally inactive. In a previous report, the inventors' laboratory demonstrated that HDAC 4 and 5 dimerize with MEF2 and repress the transcriptional activity of MEF2 and, further, that this interaction requires the presence of the N-terminus of the HDAC 4 and 5 proteins (McKinsey et al., 2000a,b).

Recently, the link between HDAC's and MEF2 has been unraveled and described (McKinsey et al., 2002). It was shown that the association between HDAC's and MEF2 is controlled by phosphorylation, and that a kinase that was as then unidentified mediated this association. Mutant HDAC's lacking phosphorylation sites acted as signal-resistant repressors to cardiomyocyte hypertrophy and HDAC knock out mice were hypersensitive to heart failure and hypertrophy (Zhang et al., 2002). It has also has been shown that certain HDAC inhibitors are anti-hypertrophic. In other contexts, recent research has also highlighted the important role of HDACs in cancer biology. In fact, various inhibitors of HDACs are being tested for their ability to induce cellular differentiation and/or apoptosis in cancer cells (Marks et al., 2000). Such inhibitors include suberoylanilide hydroxamic acid (SAHA) (Butler et al., 2000; Marks et al., 2001), m-carboxycinnamic acid bis-hydroxamide (Coffey et al., 2001) and pyroxamide (Butler et al., 2001).

All of these findings demonstrate the important role of HDAC's in disease progression, and specific data demonstrates that the HDAC-MEF2 association is a key factor in cardiac disease. Thus, the kinase responsible for mediating this association is a convergence point for the cascade leading to hypertrophy and is a potential therapeutic target. To date, the kinase responsible for this association has not been identified.

### SUMMARY OF THE INVENTION

Thus, in accordance with the present invention, there is provided a method of treating pathologic cardiac hypertrophy and heart failure comprising (a) identifying a patient having cardiac hypertrophy or heart failure; and (b) administering to the patient an inhibotor of PKD. Administering may comprise intravenous, oral, transdermal, sustained release, delayed release, controlled release, suppository, sublingual administration, or by direct injection into cardiac tissue.

The method may further comprise administering a second therapeutic regimen, such as a beta blocker, an ionotrope, a diuretic, ACE-I, AII antagonist, BNP, Ca⁺⁺-blocker, or an HDAC inhibitor. The second therapeutic regimen may be administered at the same time as the inhibitor of PKD, or either before or after the inhibitor of PKD. The treatment may improve one or more symptoms of pathologic cardiac hypertrophy or heart failure such as providing increased exercise capacity, increased cardiac ejection volume, decreased left ventricular end diastolic pressure, decreased pulmonary capillary wedge pressure, increased cardiac output or cardiac index, lowered pulmonary artery pressures, decreased left ventricular end systolic and diastolic dimensions, decreased left and right ventricular wall stress, decreased wall tension and wall thickness, increased quality of life, and decreased disease-related morbidity and mortality.

In yet another embodiment, there is provided a method of preventing pathologic cardiac hypertrophy or heart failure comprising (a) identifying a patient at risk of developing pathologic cardiac hypertrophy or heart failure; and (b) administering to the patient an inhibitor of PKD. Administration may comprise intravenous, oral, transdermal, sustained release, delayed release, controlled release, suppository, sublingual administration, or direct injection into cardiac tissue. The patient at risk may exhibit one or more of a list of risk factors comprising long standing uncontrolled hypertension, uncorrected valvular disease, chronic angina, or recent myocardial infarction. The patient at risk my also have a congenital, familiar, or genetic predisposition to heart disease, heart failure or cardiac hypertrophy. Heart failure or symptoms thereof may comprise ischemia, cardiomyopathy, aortic stenosis, or other heart muscle diseases.

In accordance with the preceding embodiments, the inhibitor of PKD may be any molecule that effects a reduction in the activity of PKD or inhibits PKD's phosphorylation of class-II HDAC's. This includes proteins, peptides, DNA molecules (including antisense), RNA molecules (including RNAi, antisense, and ribozymes), antibodies (including single chain antibodies), expression constructs that encode antibodies, and small molecules. The small molecules may include, but are not limited to, resveratrol, indolocarbazoles, Godecke 6976 (Go6976), staurosporine, K252a, Substance P (SP) analogues including [d-Arg(1),d-Trp(5,7,9), Leu(11)]SP, PKC inhibitor 109203X (GF-1), PKC inhibitor Ro 31-8220, GO 7874, Genistein, the specific Src inhibitors PP-1 and PP-2, chelerythrine, or rottlerin.

HDAC inhibitors may include, but are not limited to, trichostatin A, trapoxin B, MS 275-27, m-carboxycinnamic acid bis-hydroxamide, depudecin, oxamflatin, apicidin, suberoylanilide hydroxamic acid, Scriptaid, pyroxamide, 2-amino-8-oxo-9,10-epoxy-decanoyl, 3-(4-aroyl-1H-pyrrol-2-yl)-N-hydroxy-2-propenamide and FR901228. Additionally, the following references describe histone deacetylase inhibitors which may be selected for use in the current invention: AU 9,013,101; AU 9,013,201; AU 9,013,401; AU 6,794,700; EP 1,233,958; EP 1,208,086; EP 1,174,438; EP 1,173,562; EP 1,170,008; EP 1,123,111; JP 2001/348340; U.S. 2002/103192; U.S. 2002/65282; U.S. 2002/61860; WO 02/51842; WO 02/50285; WO 02/46144; WO 02/46129; WO 02/30879; WO 02/26703; WO 02/26696; WO 01/70675; WO 01/42437; WO 01/38322; WO 01/18045; WO 01/14581; Furumai et al. (2002); Hinnebusch et al. (2002); Mai et al. (2002); Vigushin et al. (2002); Gottlicher et al. (2001); Jung (2001); Komatsu et al. (2001); Su et al. (2000).

In still another embodiment, there is provided a method of assessing the efficacy of an inhibitor of PKD in treating cardiac hypertrophy or heart failure comprising (a) providing an inhibitor of PKD; (b) treating a cell with the inhibitor of PKD; and (c) measuring the expression of one or more cardiac hypertrophy parameters, wherein a change in the one or more cardiac hypertrophy parameters, as compared to one or more cardiac hypertrophy parameters in a cell not treated with the inhibitor of PKD, identifies the inhibitor of PKD as an inhibitor of cardiac hypertrophy or heart failure.

The cell may be a myocyte, an isolated myocyte such as a cardiomyocyte, comprised in isolated intact tissue, a neonatal rat ventricular myocyte, an H9C2 cell, a cardiomyocyte located in vivo in an intact heart muscle, or part of a transgenic, non-human mammal. The myocyte or intact heart muscle may be subjected to a stimulus that triggers a hypertrophic response in the one or more cardiac hypertrophy parameters. Said stimulus may be aortic banding, rapid cardiac pacing, induced myocardial infarction, expression of a transgene, treatment with a chemical, pharmaceutical, or drug. The chemical or pharmaceutical agent may include, but is not limited to, angiotensin II, isopreterenol, phenylephrine, endothelin-I, vasoconstrictors, and antidiuretics. The treating may be performed in vivo or in vitro.

The one or more cardiac hypertrophy parameters may comprise right ventricular ejection fraction, left ventricular ejection fraction, ventricular wall thickness, heart weight/body weight ratio, right or left ventricular weight/body weight ratio, or cardiac weight normalization measurement. Said parameters may further comprise the expression level of one or more target genes in said myocyte or intact heart muscle, wherein expression level of said one or more target genes is indicative of cardiac hypertrophy.

The one or more target genes may be selected from the group consisting of ANF, α-MyHC, β-MyHC, α-skeletal actin, cardiac actin, SERCA, cytochrome oxidase subunit VIII, mouse T-complex protein, insulin growth factor binding protein, Tau-microtubule-associated protein, ubiquitin carboxyl-terminal hydrolase, Thy-1 cell-surface glycoprotein, or MyHC class I antigen. The expression level may be measured using a reporter protein coding region operably linked to a target gene promoter, such as luciferase, β-gal or green fluorescent protein. The expression level may be measured using hybridization of a nucleic acid probe to a target mRNA or amplified nucleic acid product.

The one or more cardiac hypertrophy parameters also may comprise one or more aspects of cellular morphology, such as sarcomere assembly, cell size, cell contractility, total protein synthesis, or cell toxicity. The cell may further express a mutant class-II HDAC protein lacking one or more phosphorylation sites, wherein measuring comprises measuring the phosphorylation of class-II HDAC's, nuclear export of class-II HDAC's, or the association of class-II HDAC's with MEF-2. Measuring may further comprise measuring for an enhancement of class-II HDAC association with MEF-2 or for an decrease in MEF-2 dependent transcription.

In still yet another embodiment, there is provided a method of identifying inhibitors of cardiac hypertrophy or heart failure comprising (a) providing a PKD; (b) contacting the PKD with a candidate inhibitor substance; and (c) measuring the activity of the PKD, wherein a greater decrease in the kinase activity of the PKD identifies the candidate inhibitor substance as an inhibitor of cardiac hypertrophy or heart failure. The PKD may be purified away from whole cells or heart cells, or it can be located in an intact cell. The cell may be a myocyte, such as a cardiomyocyte.

The decrease in kinase activity may be measured by a decrease in phosphorlyation of HDAC, more specifically a class-II HDAC. The candidate inhibitor substance may include, but is not limited to, an interfering RNA, an antibody preparation, a single chain antibody, an RNA or DNA antisense construct, an enzyme, a chemical, drug, or pharmaceutical, or small molecule. The candidate inhibitor may further be resveratrol, indolocarbazoles, Godecke 6976 (Go6976), staurosporine, K252a, Substance P (SP) analogues including [d-Arg(1),d-Trp(5,7,9), Leu(11)]SP, PKC inhibitor 109203X (GF-1), PKC inhibitor Ro 31-8220, GO 7874, Genistein, the specific Src inhibitors PP-1 and PP-2, chelerythrine, or rottlerin.

The decrease in kinase activity may further be measured by measuring an inhibition of PKD's binding to class-II HDAC by co-immunoprecipitation, or by measuring a block in phosphorylation of class-II HDAC's. The inhibitor of PKD may enhance the association of class-II HDAC's with MEF-2 or other class-II HDAC regulated transcription factors.

In a further embodiment of the invention, there is provided a transgenic, non-human mammal, the cells of which comprise a heterologous PKD gene under the control of a promoter active in eukaroyic cells. In another embodiment there is provided a transgenic, non-human mammal, the cells of which comprise a PKD gene under the control of a heterologous promoter active in the cells of said non-human mammal. In yet a further embodiment, there is provided a transgenic, non-human mammal, the cells of which lack one or both native PKD alleles, and these alleles may further have been knocked out by homologous recombination. The transgenic, non-human mammal may be a mouse. The promoter may be tissue specific, and may further be specific to muscle tissue, and may yet further be specific to heart muscle tissue.

The PKD gene may be human. The gene may further encode a constitutively active form of the protein or a dominant negative version of the protein. The promoter may be active in eukaryotic cells. The muscle specific promoter may be selected from the group consisting of myosin light chain-2 promoter, alpha actin promoter, troponin 1 promoter, Na+/Ca2+ exchanger promoter, dystrophin promoter, creatine kinase promoter, alpha7 integrin promoter, brain natriuretic peptide promoter, myosoin heavy chain promoter, ANF promoter, and alpha B-crystallin/small heat shock protein promoter.

In yet further embodiments of the invention, decreasing PKD activity in the heart cells of a subject is offered as a treatment for myocardial infract, prevention of cardiac hypertrophy and dilated cardiomyopathy, inhibition of progression of cardiac hypertrophy, treatment of heart failure, inhibition of progression of heart failure, increasing exercise tolerance in a subject with heart failure or cardiac hypertrophy, reducing hospitalization in a subject with heart failure or cardiac hypertrophy, improving quality of life in a subject with heart failure or cardiac hypertrophy, and decreasing morbidity or mortality in subjects with heart failure or cardiac hypertrophy.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D—PKC-dependent nuclear export of HDAC5. FIG. 1A. COS cells were cultured on 6-well dishes, transfected with a GFP-HDAC5 expression vector (1 μg) and stimulated with the indicated compounds, as described in Materials and Methods. Sixty minutes after addition of compounds, GFP-HDAC5 distribution was determined by fluorescence microscopy. PMA stimulation resulted in complete relocalization of GFP-HDAC5 from the nucleus to the cytoplasm, while ionomycin triggered a partial response. FIG. 1B. COS cells were transfected with expression vectors encoding FLAG-tagged versions of either HDAC5 or an HDAC5 mutant harboring alanines in place of serines 259 and 498 (HDAC5 S259/498A) (1 μg each). Cells were stimulated with PMA for 60 min and HDAC5 distribution was determined by indirect immunofluorescence with anti-FLAG primary antibody and fluorecein-conjugated secondary antibody. HDAC5 259/498A is refractory to PMA stimulation. FIG. 1C. COS cells transfected with GFPHDAC5 encoding expression vector (1 μg) and stimulated with PMA for the indicated times. FIG. 1D. COS cells were transiently transfected with expression vectors encoding FLAG-tagged versions of HDAC5 or HDAC5 S259/498A (1 μg each). Cells were pretreated with the PKC inhibitor bisindolylmaleimide (Bis I; 10 μM) for 30 minutes and stimulated with PMA for 30 min, as indicated. Association of FLAG-HDAC5 with endogenous 14-3-3 was detected by sequential immunoprecipitation and immunoblotting.

FIGS. 2A-D—PKC inhibition blocks PE-mediated nuclear export of HDAC5 in cardiomyocytes. FIG. 2A. Schematic representation of a quantitative assay for HDAC5 nuclear export. NVRMs are cultured in 96-well dishes and infected with adenovirus encoding GFP-HDAC5. Cells are serum-starved, subjected to agonists and inhibitors, fixed and stained with Hoechst dye. Relative abundance of GFP-HDAC5 in the nucleus versus the cytoplasm is quantified employing the Cellomics High Content Imaging System, which demarcates nuclei based on Hoechst fluorescence and defines a cytoplasmic ring based on these nuclear dimensions. Values represent the mean of nuclear minus cytoplasmic fluorescence intensity difference. FIG. 2B. Assay validation. NRVMs were infected with denovirus encoding GFP-HDAC5 and exposed to PE ranging in concentration form 0.1 to 20 μM. Cells were prepared for Cellomics analysis following 2 hrs of stimulation. Mean nuclear minus cytoplasmic fluorescenceintensity difference was determined for at least 50 cells/well in 8 wells/condition (400 cells total). The value for untreated cells was set to 100%. PE triggered dose-dependent nuclear export of HDAC5. FIG. 2C. NRVMs were infected with adenoviral GFP-HDAC5 and pre-treated with kinase inhibitors (concentrations of inhibitors are described in Materials and Methods). Subcellular distribution of HDAC5 was quantified following stimulation with PE (20 μM) for 2 hrs. Mean nuclear minus cytoplasmic fluorescence intensity difference was determined for at least 50 cells/well in 8 wells/condition (400 cells total). Higher values indicate greater abundance of HDAC5 in the nucleus. Well-to-well standard deviations are shown. Only staurosporine and the PKC inhibitor Bis I were effective in blocking HDAC5 nuclear export. Representative images are shown in FIG. 2D.

FIGS. 3A-C—Inhibition of PKC-mediated cardiac hypertrophy by signalresistant HDAC5. NRVMs were cultured on 6-well dishes and infected with adenoviruses (MOI=10) encoding a LacZ control (Ad-LacZ) or FLAG-tagged HDAC5 harboring alanines in place of serines 259 and 498 (Ad-HDAC5 S/A), which are required for 14-3-3-mediated nuclear export. Cells were treated with PE (20 μM) or PMA (100 nM) for 24 hrs prior to analysis. FIG. 3A. Cells were fixed and sarcomeres visualized by indirect immunofluorescence with primary antibody specific for α-actinin and fluorescein-conjugated secondary antibody. FIG. 3B. ANF protein was detected by indirect immunofluorescence with anti-ANF primary antibody. FIG. 3C. Total RNA was harvested from cells and subjected to dot blot analysis with radiolabeled oligonucleotides specific for the indicated transcripts. RNA levels were quantified using a phosphorimager and are depicted as -fold change relative to amounts in unstimulated cells infected with Ad-LacZ. Values were normalized to GAPDH controls.

FIGS. 4A-F—Differential requirement for PKC in agonist-mediated nuclear export of HDAC5. FIG. 4A. NRVMs were cultured on 96-well dishes and infected with adenoviral GFP-HDAC5, as described in above. Cells were serum-starved for four hours prior to stimulation with PE (20 μM), ET-1 (50 nM) or FBS (10%) for two hours. FIG. 4B. Cells were prepared as described in FIG. 4A. Following serum starvation, infected NRVMs were pretreated with Bis I (10 μM) for 30 minutes and stimulated with the indicated agonists for 2 hours. Nuclear export of HDAC5 was quantified using the Cellomics Imaging System, as described above. Higher values indicate greater abundance of HDAC5 in the nucleus. FIG. 4C. Experiment was performed as described in FIG. 4B, except that cells received Gö6983 (10 μM) prior to agonists. FIG. 4D. Experiment was performed as described in FIG. 4B, except that cells received increasing doses of Gö6976 prior to agonists. FIG. 4E. Representative images from each treatment group were captured using a fluorescence microscope equipped with a digital camera. FIG. 4F. NRVMs were infected with adenovirus encoding GFP-HDAC5 and cultured on 10-cm dishes. Twenty-fours hrs post-infection, cells were serum-starved for four hours and pre-treated with Bis I (10 μM) or Gö6976 (10 μM) for one hour prior to stimulation with PE (20 μM) or ET-1 (50 nM) for one hour. Whole-cell protein lysates were prepared and subjected to sequential immunoprecipitation and immunoblotting, as indicated.

FIGS. 5A-E—Protein kinase D is an HDAC5 kinase. FIG. 5A. Amino acid sequences surrounding the regulatory phosphorylation sites of class II HDACs. NLS: nuclear localization signal; HDAC domain: deacetylase catalytic domain. The consensus target site for PKD is shown. Leucine at position −5 relative the phosphorylation site is required for optimal PKD-directed phosphorylation of other proteins. FIG. 5B. COS cells were transfected with an expression vector encoding GFP fused to HDAC5 harboring glycines in place of leucines 254 and 493 (L254/493G). Twenty-four hrs post-transfection, cells were left untreated (control) or stimulated with PMA for 30 minutes. FIG. 5C. COS cells were cotransfected with expression vectors (1 μg each) encoding GFP-HDAC5 or GFPHDAC5 S/A and constitutively active (S/E) or catalytically inactive (K/W) forms of PKD. HDAC5 localization was determined 24 hrs post-transfection. FIG. 5D. COS cells were co-transfected with expression vectors (1 μg each) encoding FLAGHDAC5 and HA-tagged versions of either wild-type, constitutively active (S/E), or catalytically inactive (K/W) PKD. Twenty-four hrs post-transfection, cells were treated with PMA or vehicle control for 30 min. FLAG-HDAC5 was immunoprecipitated from whole-cell protein lysates and either incorporated into an in vitro kinase assay (IVK) or resolved by SDS-PAGE for western blot analysis to detect associated PKD, as indicated. Phosphorylated HDAC5 was resolved by SDS-PAGE and detected by autoradiography. FIG. 5E. Mammalian two-hybrid assay. Expression vectors encoding the GAL4 DNA binding domain fused to HDAC5 (Gal4-HDAC5) or the indicated HDAC5 alanine substitution mutants were co-transfected into COS cells with a plasmid encoding 14-3-3 fused to the VP16 transcriptional activation domain (14-3-3-VP16), a Gal4-dependent luciferase reporter, and a vector encoding constitutively active PKD (S/E). PKD stimulates association between HDAC5 and 14-3-3, which is dependent on the phospho-acceptors at positions 259 and 498.

FIGS. 6A-B—Association of endogenous PKD with HDAC5 in cardiomyocytes. FIG. 6A. NRVMs were cultured on 10-cm dishes and infected with adenovirus encoding FLAG-HDAC5. Twenty-four hrs post-transfection, cells were stimulated with PMA for 30 min and whole-cell protein lysates prepared. Some cells were pre-treated with Bis I (pre-Bis I; 10 μM) for 30 min prior to PMA stimulation. FLAG-HDAC5 was immunoprecipitated and incorporated into in vitro kinase reactions supplemented with Bis I (post-Bis I; 10 μM) or Gö6976 (post-Gö6976; 10 μM), as indicated. Phosphorylation of HDAC5 was blocked when cells were pre-treated with Bis I (pre-Bis I). Gö6976, but not Bis I, blocked phosphoryl transfer to HDAC5 when added directly to kinase reaction mixtures. FIG. 6B. NRVMs were infected with adenovirus encoding GFP-HDAC5 and cultured on 10-cm dishes. Twenty-four hrs post-infection, cells were serum-starved for 4 hrs, and pre-treated with Bis I (10 μM) for 30 min prior to stimulation with PE (20 μM) for 1 hr. HDAC5 was immunoprecipitated from whole-cell lysates and associated total PKD or PKD auto-phosphorylated at serine 916 (p-916) were detected by immunoblotting. Blots were reprobed with GFP-specific antibodies to determine total amounts immunoprecipitated HDAC5.

FIGS. 7A-D—Cardiac expression of activated PKD leads to dilated cardiomyopathy. FIG. 7A. Transgenic mice expressing constitutively active PKD under the control of the cardiac-specific α-myosin heavy chain (αMHC) promoter were generated as described in Materials and Methods. Shown are H&E sections of wild-type and aMHC-PKD transgenic hearts at 4 weeks of age. Also shown is an aMHC-PKD transgenic heart at 4 months displaying a dilated cardiomyopathy. FIG. 7B. Heart weight to body ratios of wild-type and aMHC-PKD transgenic mice at 4 weeks of age. FIG. 7C. Western blot analysis of lysates from wild-type, aMHC-PKD, and aMHC-calcineurin (CnA) hearts. Total and activated (P-916) PKD protein levels were measured. The arrowhead marks the band corresponding to PKD1. FIG. 7D. Dot blot analysis for fetal gene markers was performed on total RNA from hearts of 4 week old wild-type, CnA, or PKD transgenic mice. The numbers below the CnA and PKD blots represent fold increases versus wild-type samples after normalization to GAPDH levels.

FIG. 8—A model of kinase-dependent signaling pathways that regulate nuclear export of class II HDACs and cardiac hypertrophy. Stimulation of cardiac myocyte hypertrophy by the α-adrenergic agonists phenylephrine (PE) or endothelin-1 (ET-1) leads to phosphorylation and nuclear export of HDACs through activation of PKD. Activation of PKD by PE occurs through a PKC dependent pathway, primarily the calcium-independent novel PKCs (nPKC). However, activation of PKD by ET-1 in cardiomyocytes appears to be PKCindependent. Subsequent phosphorylation of HDAC5 by PKD leads to its nuclear export through association with 14-3-3 and activation of MEF2 and thehypertrophic genetic program.

### DETAILED DESCRIPTION OF ILLUSTRATIVE EMBODIMENTS

Heart failure is one of the leading causes of morbidity and mortality in the world. In the U.S. alone, estimates indicate that 3 million people are currently living with cardiomyopathy and another 400,000 are diagnosed on a yearly basis. Dilated cardiomyopathy (DCM), also referred to as “congestive cardiomyopathy,” is the most common form of the cardiomyopathies and has an estimated prevalence of nearly 40 per 100,000 individuals (Durand et al., 1995). Although there are other causes of DCM, familiar dilated cardiomyopathy has been indicated as representing approximately 20% of “idiopathic” DCM. Approximately half of the DCM cases are idiopathic, with the remainder being associated with known disease processes. For example, serious myocardial damage can result from certain drugs used in cancer chemotherapy (e.g., doxorubicin and daunoribucin). In addition, many DCM patients are chronic alcoholics. Fortunately, for these patients, the progression of myocardial dysfunction may be stopped or reversed if alcohol consumption is reduced or stopped early in the course of disease. Peripartum cardiomyopathy is another idiopathic form of DCM, as is disease associated with infectious sequelae. In sum, cardiomyopathies, including DCM, are significant public health problems.

Heart disease and its manifestations, including coronary artery disease, myocardial infarction, congestive heart failure and cardiac hypertrophy, clearly presents a major health risk in the United States today. The cost to diagnose, treat and support patients suffering from these diseases is well into the billions of dollars. Two particularly severe manifestations of heart disease are myocardial infarction and cardiac hypertrophy. With respect to myocardial infarction, typically an acute thrombocytic coronary occlusion occurs in a coronary artery as a result of atherosclerosis and causes myocardial cell death. Because cardiomyocytes, the heart muscle cells, are terminally differentiated and generally incapable of cell division, they are generally replaced by scar tissue when they die during the course of an acute myocardial infarction. Scar tissue is not contractile, fails to contribute to cardiac function, and often plays a detrimental role in heart function by expanding during cardiac contraction, or by increasing the size and effective radius of the ventricle, for example, becoming hypertrophic. With respect to cardiac hypertrophy, one theory regards this as a disease that resembles aberrant development and, as such, raises the question of whether developmental signals in the heart can contribute to hypertrophic disease. Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to DCM, heart failure, and sudden death. In the United States, approximately half a million individuals are diagnosed with heart failure each year, with a mortality rate approaching 50%.

The causes and effects of cardiac hypertrophy have been extensively documented, but the underlying molecular mechanisms have not been elucidated. Understanding these mechanisms is a major concern in the prevention and treatment of cardiac disease and will be crucial as a therapeutic modality in designing new drugs that specifically target cardiac hypertrophy and cardiac heart failure. As pathologic cardiac hypertrophy typically does not produce any symptoms until the cardiac damage is severe enough to produce heart failure, the symptoms of cardiomyopathy are those associated with heart failure. These symptoms include shortness of breath, fatigue with exertion, the inability to lie flat without becoming short of breath (orthopnea), paroxysmal nocturnal dyspnea, enlarged cardiac dimensions, and/or swelling in the lower legs. Patients also often present with increased blood pressure, extra heart sounds, cardiac murmurs, pulmonary and systemic emboli, chest pain, pulmonary congestion, and palpitations. In addition, DCM causes decreased ejection fractions (i.e., a measure of both intrinsic systolic function and remodeling). The disease is further characterized by ventricular dilation and grossly impaired systolic function due to diminished myocardial contractility, which results in dilated heart failure in many patients. Affected hearts also undergo cell/chamber remodeling as a result of the myocyte/myocardial dysfunction, which contributes to the “DCM phenotype.” As the disease progresses so do the symptoms. Patients with DCM also have a greatly increased incidence of life-threatening arrhythmias, including ventricular tachycardia and ventricular fibrillation. In these patients, an episode of syncope (dizziness) is regarded as a harbinger of sudden death.

Diagnosis of dilated cardiomyopathy typically depends upon the demonstration of enlarged heart chambers, particularly enlarged ventricles. Enlargement is commonly observable on chest X-rays, but is more accurately assessed using echocardiograms. DCM is often difficult to distinguish from acute myocarditis, valvular heart disease, coronary artery disease, and hypertensive heart disease. Once the diagnosis of dilated cardiomyopathy is made, every effort is made to identify and treat potentially reversible causes and prevent further heart damage. For example, coronary artery disease and valvular heart disease must be ruled out. Anemia, abnormal tachycardias, nutritional deficiencies, alcoholism, thyroid disease and/or other problems need to be addressed and controlled.

As mentioned above, treatment with pharmacological agents still represents the primary mechanism for reducing or eliminating the manifestations of heart failure. Diuretics constitute the first line of treatment for mild-to-moderate heart failure. Unfortunately, many of the commonly used diuretics (e.g., the thiazides) have numerous adverse effects. For example, certain diuretics may increase serum cholesterol and triglycerides. Moreover, diuretics are generally ineffective for patients suffering from severe heart failure.

If diuretics are ineffective, vasodilatory agents may be used; the angiotensin converting (ACE) inhibitors (e.g., enalopril and lisinopril) not only provide symptomatic relief, they also have been reported to decrease mortality (Young et al., 1989). Again, however, the ACE inhibitors are associated with adverse effects that result in their being contraindicated in patients with certain disease states (e.g., renal artery stenosis). Similarly, inotropic agent therapy (i.e., a drug that improves cardiac output by increasing the force of myocardial muscle contraction) is associated with a panoply of adverse reactions, including gastrointestinal problems and central nervous system dysfunction.

Thus, the currently used pharmacological agents have severe shortcomings in particular patient populations. The availability of new, safe and effective agents would undoubtedly benefit patients who either cannot use the pharmacological modalities presently available, or who do not receive adequate relief from those modalities. The prognosis for patients with DCM is variable, and depends upon the degree of ventricular dysfunction, with the majority of deaths occurring within five years of diagnosis.

I. The Present Invention

The inventors have shown previously that MEF2 is activated by MAP kinase phosphorylation of three conserved sites in its carboxy-terminal activation domain (see, Katoh et al. 1998). CaMK signaling also activates MEF2 by phosphorylating the class II HDACs, which are expressed at high levels in the adult heart where they can repress MEF2 activity. Upon phosphorylation, these HDACs bind to 14-3-3, and dissociate from MEF2, with resulting translocation to the nucleus and activation of MEF2-dependent transcription. Mutants of class II HDACs that cannot be phosphorylated cannot detach from MEF2 and irreversibly block expression of MEF2 target genes.

It has also been shown that an adenovirus encoding a non-phosphorylatable mutant of HDAC 5 is capable of preventing cardiomyocyte hypertrophy in vitro in response to diverse signaling pathways (Lu et al., 2000). These findings suggest that phosphorylation of these conserved sites in class II HDACs is an essential step for initiating cardiac hypertrophy. They further suggest that inhibiting the phosphorylation of class-II HDACs by isolating and targeting the kinase responsible for phosphorylating class-II HDAC's, would block hypertrophy and subsequent development of heart failure.

The inventors herein describe the characterization of PKD as the kinase that is responsible for phosphorylating class-II HDAC's, mediating their interaction with MEF-2, and in part controlling the nuclear or cytoplasmic localization of class-II HDAC's. The present invention also presents a therapeutic intervention in cardiac hypertrophy and heart failure through inhibition of PKD, as well as tools for screening for therapeutics for the treatment of cardiac hypertrophy and heart failure.

II. Protein Kinases

Kinases regulate many different cell proliferation, differentiation, and signaling processes by adding phosphate groups to proteins. Uncontrolled signaling has been implicated in a variety of disease conditions including inflammation, cancer, arteriosclerosis, psoriasis, and heart disease and hypertrophy. Reversible protein phosphorylation is the main strategy for controlling activities of eukaryotic cells. It is estimated that more than 1000 of the 10,000 proteins active in a typical mammalian cell are phosphorylated. The high energy phosphate, which drives activation, is generally transferred from adenosine triphosphate molecules (ATP) to a particular protein by protein kinases and removed from that protein by protein phosphatases. Phosphorylation occurs in response to extracellular signals (hormones, neurotransmitters, growth and differentiation factors, etc.), cell cycle checkpoints, and environmental or nutritional stresses and is roughly analogous to turning on a molecular switch. When the switch goes on, the appropriate protein kinase activates a metabolic enzyme, regulatory protein, receptor, cytoskeletal protein, ion channel or pump, or transcription factor.

The kinases comprise the largest known protein group, a superfamily of enzymes with widely varied functions and specificities. They are usually named after their substrate, their regulatory molecules, or some aspect of a mutant phenotype. With regard to substrates, the protein kinases may be roughly divided into two groups; those that phosphorylate tyrosine residues (protein tyrosine kinases, PTK) and those that phosphorylate serine or threonine residues (serine/threonine kinases, STK). A few protein kinases have dual specificity and phosphorylate threonine and tyrosine residues. Almost all kinases contain a similar 250-300 amino acid catalytic domain. The N-terminal domain, which contains subdomains I-IV, generally folds into a two-lobed structure, which binds and orients the ATP (or GTP) donor molecule. The larger C terminal lobe, which contains subdomains VI A-XI, binds the protein substrate and carries out the transfer of the gamma phosphate from ATP to the hydroxyl group of a serine, threonine, or tyrosine residue. Subdomain V spans the two lobes.

The kinases may be categorized into families by the different amino acid sequences (generally between 5 and 100 residues) located on either side of, or inserted into loops of, the kinase domain. These added amino acid sequences allow the regulation of each kinase as it recognizes and interacts with its target protein. The primary structure of the kinase domains is conserved and can be further subdivided into 11 subdomains. Each of the 11 subdomains contains specific residues and motifs or patterns of amino acids that are characteristic of that subdomain and are highly conserved (Hardie and Hanks, 1995).

The second messenger dependent protein kinases primarily mediate the effects of second messengers such as cyclic AMP (cAMP), cyclic GMP, inositol triphosphate, phosphatidylinositol, 3,4,5-triphosphate, cyclic-ADPribose, arachidonic acid, diacylglycerol and calcium-calmodulin. The cyclic-AMP dependent protein kinases (PKA) are important members of the STK family. Cyclic-AMP is an intracellular mediator of hormone action in all prokaryotic and animal cells that have been studied. Such hormone-induced cellular responses include thyroid hormone secretion, cortisol secretion, progesterone secretion, glycogen breakdown, bone resorption, and regulation of heart rate and force of heart muscle contraction. PKA is found in all animal cells and is thought to account for the effects of cyclic-AMP in most of these cells. Altered PKA expression is implicated in a variety of disorders and diseases including cancer, thyroid disorders, diabetes, atherosclerosis, and cardiovascular disease (Isselbacher et al., 1994).

Calcium-calmodulin (CaM) dependent protein kinases are also members of STK family. Calmodulin is a calcium receptor that mediates many calcium regulated processes by binding to target proteins in response to the binding of calcium. The principle target protein in these processes is CaM dependent protein kinases. CaM-kinases are involved in regulation of smooth muscle contraction (MLC kinase), glycogen breakdown (phosphorylase kinase), and neurotransmission (CaM kinase I and CaM kinase II). CaM kinase I phosphorylates a variety of substrates including the neurotransmitter related proteins synapsin I and II, the gene transcription regulator, CREB, and the cystic fibrosis conductance regulator protein, CFTR (Haribabu et al., 1995). CaM II kinase also phosphorylates synapsin at different sites, and controls the synthesis of catecholamines in the brain through phosphorylation and activation of tyrosine hydroxylase. Many of the CaM kinases are activated by phosphorylation in addition to binding to CaM. The kinase may autophosphorylate itself, or be phosphorylated by another kinase as part of a “kinase cascade.”

Another ligand-activated protein kinase is 5′-AMP-activated protein kinase (AMPK) (Gao et al., 1996). Mammalian AMPK is a regulator of fatty acid and sterol synthesis through phosphorylation of the enzymes acetyl-CoA carboxylase and hydroxymethylglutaryl-CoA reductase and mediates responses of these pathways to cellular stresses such as heat shock and depletion of glucose and ATP. AMPK is a heterotrimeric complex comprised of a catalytic alpha subunit and two non-catalytic beta and gamma subunits that are believed to regulate the activity of the alpha subunit. Subunits of AMPK have a much wider distribution in non-lipogenic tissues such as brain, heart, spleen, and lung than expected. This distribution suggests that its role may extend beyond regulation of lipid metabolism alone.

The mitogen-activated protein kinases (MAP) are also members of the STK family. MAP kinases also regulate intracellular signaling pathways. They mediate signal transduction from the cell surface to the nucleus via phosphorylation cascades. Several subgroups have been identified, and each manifests different substrate specificities and responds to distinct extracellular stimuli (Egan and Weinberg, 1993). MAP kinase signaling pathways are present in mammalian cells as well as in yeast. The extracellular stimuli that activate mammalian pathways include epidermal growth factor (EGF), ultraviolet light, hyperosmolar medium, heat shock, endotoxic lipopolysaccharide (LPS), and pro-inflammatory cytokines such as tumor necrosis factor (TNF) and interleukin-1 (IL-1).

PRK (proliferation-related kinase) is a serum/cytokine inducible STK that is involved in regulation of the cell cycle and cell proliferation in human megakaroytic cells (Li et al., 1996). PRK is related to the polo (derived from humans polo gene) family of STKs implicated in cell division. PRK is downregulated in lung tumor tissue and may be a proto-oncogene whose deregulated expression in normal tissue leads to oncogenic transformation. Altered MAP kinase expression is implicated in a variety of disease conditions including cancer, inflammation, immune disorders, and disorders affecting growth and development.

The cyclin-dependent protein kinases (CDKs) are another group of STKs that control the progression of cells through the cell cycle. Cyclins are small regulatory proteins that act by binding to and activating CDKs that then trigger various phases of the cell cycle by phosphorylating and activating selected proteins involved in the mitotic process. CDKs are unique in that they require multiple inputs to become activated. In addition to the binding of cyclin, CDK activation requires the phosphorylation of a specific threonine residue and the dephosphorylation of a specific tyrosine residue.

Protein tyrosine kinases, PTKs, specifically phosphorylate tyrosine residues on their target proteins and may be divided into transmembrane, receptor PTKs and nontransmembrane, non-receptor PTKs. Transmembrane protein-tyrosine kinases are receptors for most growth factors. Binding of growth factor to the receptor activates the transfer of a phosphate group from ATP to selected tyrosine side chains of the receptor and other specific proteins. Growth factors (GF) associated with receptor PTKs include; epidermal GF, platelet-derived GF, fibroblast GF, hepatocyte GF, insulin and insulin-like GFs, nerve GF, vascular endothelial GF, and macrophage colony stimulating factor.

Non-receptor PTKs lack transmembrane regions and, instead, form complexes with the intracellular regions of cell surface receptors. Such receptors that function through non-receptor PTKs include those for cytokines, hormones (growth hormone and prolactin) and antigen-specific receptors on T and B lymphocytes.

Many of these PTKs were first identified as the products of mutant oncogenes in cancer cells where their activation was no longer subject to normal cellular controls. In fact, about one third of the known oncogenes encode PTKs, and it is well known that cellular transformation (oncogenesis) is often accompanied by increased tyrosine phosphorylation activity (Carbonneau and Tonks, 1992). Regulation of PTK activity may therefore be an important strategy in controlling some types of cancer.

A. Protein Kinase C Family

Protein kinase C (PKC) proteins are members of the STK family. Protein kinase D (PKD) proteins bind phorbol esters and diacylglycerol and are closely related to PKCs (Valverde et al., 1994). Protein kinase C plays a key role in modulating cellular responses in a wide variety of extracellular receptor-mediated signal transduction pathways, and in regulating cellular differentiation and proliferation in a wide variety of cells.

Protein kinase C genes/proteins may play an important role in many cancers, and therefore may be useful for drug development and for screening for, diagnosing, preventing, and/or treating a variety of cancers. For example, tumor-specific deletions have been identified within the gene for alpha-type protein kinase C in a melanoma cell line (Linnenbach et al., 1988). Elevated expression levels of PKCs have been observed in certain tumor cell lines and it has been suggested that PKCs play an important role in signal transduction pathways related to growth control (Johannes et al., 1994).

Kinase proteins, particularly members of the protein kinase C subfamily, are a major target for drug action and development. Accordingly, it is valuable to the field of pharmaceutical development to identify and characterize previously unknown members of this subfamily of kinase proteins. The present invention advances the state of the art by providing a human kinase protein that has homology to members of the protein kinase C subfamily and is implicated in cardiovascular disease. The following references, hereinafter incorporated by reference, all describe protein kinase C inhibitors which may be of use in the present invention: U.S. Pat. No. 6,528,294; U.S. Pat. No. 6,441,020; U.S. Pat. No. 6,080,784; U.S. Pat. No. 6,043,270; U.S. Pat. No. 5,955,501.

B. Protein Kinase D

The PKD family comprises PKD1 (mouse PKD, human PKCμ), PKD2 and PKD3 (also named PKC.). They are members of the AGC family of serine/threonine kinases but share a unique molecular architecture that is distinct from other AGC family members. PKD1 has multiple domains: an N-terminal region with a high frequency of apolar amino acids, mainly alanine and proline, two cysteine-rich zinc-finger regions (also called C1a and C1b), a region rich in negatively charged amino acids, a pleckstrin-homology (PH) domain and a protein Ser/Thr kinase catalytic domain (Van Lint et al., 2002). A similar modular structure is found in PKD2 and PKD3.

The AGC family comprises various subclasses: cyclic-nucleotide-regulated protein kinases, PKC, PKB/Akt, G-protein-coupled receptor kinases (GRKs) and ribosomal protein S6 kinases. PKD can be classified in a novel AGC subclass since it seems to combine features of different subclasses of the family, impairing its classification in one of the existing subclasses (Van Lint et al., 2002). For example, the cysteine-rich domains of the PKD family are similar to those of the classical and novel PKCs, while the PH domain is more reminiscent of the PKB or GRK families and is not found in any PKC enzyme. The catalytic domain is structurally and functionally different from the PKC family and from other AGC family members as judged by several criteria (Hayashi et al., 1999; Nishikawa et al., 1997; Sturany et al., 2001; Valverde et al., 1994). First, compared with all other known protein kinase catalytic domains, the amino acid sequence of the PKD catalytic domain is most similar to myosin light chain kinase (MLCK) of Dictyostelium (41%) and only 30-35% similar to individual PKCs. Second, PKD1 has a unique substrate specificity: it does not favor the substrate sites with basic residues that are preferred by the PKC family but, rather, phosphorylates substrates with a leucine at position −5 relative to the serine target site (Van Lint et al., 2002). Third, PKD1 is insensitive to the PKC inhibitors GF I and Ro 31-8220. Fourth, PKD1 does not have an autoinhibitory pseudosubstrate sequence that can be found in most members of the PKC family. Therefore, although historically PKD has been classified as a PKC family member, a separate classification is more likely appropriate.

Interestingly, PKD has been shown to be regulated by 14-3-3 proteins (Van Lint et al., 2002), and HDAC's bind to 14-3-3 proteins after being phosphorylated. Furthermore, PKD has been found in adult rat heart tissue (Haworth et al., 2000). Finally, as stated above, PKD has been shown to phosphorylate substrates with a leucine at the −5 position relative to serine (Van Lint et al., 2002), which corresponds exactly with the location of the phosphorylation sites found in class-II HDAC's by the inventors (unpublished). The sequence of human PKD may be found at Accesson No. NM002742.

C. Kinase Inhibitors

As mentioned above, protein kinases constitute a significant portion of the human genome and are one of the most fundamental intracellular signalling mechanisms. Thus, control of kinase activity in a number of cells types (or lack thereof) is a major factor in many diseases, especially those involving inflammatory of proliferative responses. Despite the diversity of kinase targets (around 500 kinase sequences are known) it is only recently that drugs specifically designed to inhibit kinases have reached the market. Even though it has long been recognize that intrcellular kinase signalling was exceedingly important, only recently has enough knowledge been garnered about the nature of kinase activity and the corresponding catalytic mechanisms to allow developing safe and selective kinase inhibitor drugs. Gleevec™ (Novartis) and Iressa™ (Astra Zeneca), for example, are pioneering members of a new and exciting class of therapeutic agents which are now ripe for exploitation in the clinic. One of skill in the art will understand that there are a number of protein kinase C inhibitors and the aforementioned companies have standard methods for making and screening for these inhibitors. As such, these methods and the known kinase or compounds available for use in these systems are hereinafter incorporated by reference.

D. PKD inhibitors

Reports indicate that resveratrol may inhibit PKD (Haworth et al., 2001) and, as such, may be useful in one embodiment of this invention. Other potential inhibitors include but are not limited to indolocarbazoles, Godecke 6976 (Go6976), staurosporine, K252a, Substance P (SP) analogues including [d-Arg(1),d-Trp(5,7,9), Leu(11)]SP, PKC inhibitor 109203X (GF-1), PKC inhibitor Ro 31-8220, PKC inhibitor GO 7874, Genistein, the specific Src inhibitors PP-1 and PP-2, chelerythrine, rottlerin. In addition to the aforementioned, there are also generic, non-pharmacological methods of inhibiting genes, which are discussed below.

i. Nucleic Acids
a. Antisense Constructs

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.
ii. Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.
iii. RNAi

RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM, but concentrations of about 100 nM have achieved effective suppression of expression in mammalian cells. siRNAs have been most effective in mammalian cell culture at about 100 nM. In several instances, however, lower concentrations of chemically synthesized siRNA have been used (Caplen, et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25 mer lengths with the endogenous nuclease complex that converts long dsRNAs to siRNAs in vivo. They do not describe or present data for synthesizing and using in vitro transcribed 21-25 mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

III. Histone Deacetylase and Inhibitors

Nucleosomes, the primary scaffold of chromatin folding, are dynamic macromolecular structures, influencing chromatin solution conformations (Workman and Kingston, 1998). The nucleosome core is made up of histone proteins, H2A, HB, H3 and H4. Histone acetylation causes nucleosomes and nucleosomal arrangements to behave with altered biophysical properties. The balance between activities of histone acetyl transferases (HAT) and deacetylases (HDAC) determines the level of histone acetylation. Acetylated histones cause relaxation of chromatin and activation of gene transcription, whereas deacetylated chromatin generally is transcriptionally inactive.

Eleven different HDACs have been cloned from vertebrate organisms. The first three human HDACs identified were HDAC 1, HDAC 2 and HDAC 3 (termed class I human HDACs), and HDAC 8 (Van den Wyngaert et al., 2000) has been added to this list. Recently class II human HDACs, HDAC 4, HDAC 5, HDAC 6, HDAC 7, HDAC 9, and HDAC 10 (Kao et al., 2000) have been cloned and identified (Grozinger et al., 1999; Zhou et al. 2001; Tong et al., 2002). Additionally, HDAC 11 has been identified but not yet classified as either class I or class II (Gao et al., 2002). All share homology in the catalytic region. HDACs 4, 5, 7, 9 and 10 however, have a unique amino-terminal extension not found in other HDACs. This amino-terminal region contains the MEF2-binding domain. HDACs 4, 5 and 7 have been shown to be involved in the regulation of cardiac gene expression and in particular embodiments, repressing MEF2 transcriptional activity. The exact mechanism in which class II HDAC's repress MEF2 activity is not completely understood. One possibility is that HDAC binding to MEF2 inhibits MEF2 transcriptional activity, either competitively or by destabilizing the native, transcriptionally active MEF2 conformation. It also is possible that class II HDAC's require dimerization with MEF2 to localize or position HDAC in a proximity to histones for deacetylation to proceed.

A variety of inhibitors for histone deacetylase have been identified. The proposed uses range widely, but primarily focus on cancer therapy. Saunders et al. (1999); Jung et al. (1997); Jung et al. (1999); Vigushin et al. (1999); Kim et al. (1999); Kitazomo et al. (2001); Vigusin et al. (2001); Hoffmann et al. (2001); Kramer et al. (2001); Massa et al. (2001); Komatsu et al. (2001); Han et al. (2001). Such therapy is the subject of an NIH sponsored Phase I clinical trial for solid tumors and non-Hodgkin's lymphoma. HDAC's also increase transcription of transgenes, thus constituting a possible adjunct to gene therapy. Yamano et al. (2000); Su et al. (2000).

HDACs can be inhibited through a variety of different mechanisms—proteins, peptides, and nucleic acids (including antisense and RNAi molecules). Methods are widely known to those of skill in the art for the cloning, transfer and expression of genetic constructs, which include viral and non-viral vectors, and liposomes. Viral vectors include adenovirus, adeno-associated virus, retrovirus, vaccina virus and herpesvirus.

Also contemplated are small molecule inhibitors. Perhaps the most widely known small molecule inhibitor of HDAC function is Trichostatin A, a hydroxamic acid. It has been shown to induce hyperacetylation and cause reversion of ras transformed cells to normal morphology (Taunton et al., 1996) and induces immunsuppression in a mouse model (Takahashi et al., 1996). It is commercially available from BIOMOL Research Labs, Inc., Plymouth Meeting, Pa.

The following references, incorporated herein by reference, all describe HDAC inhibitors that may find use in the present invention: AU 9,013,101; AU 9,013,201; AU 9,013,401; AU 6,794,700; EP 1,233,958; EP 1,208,086; EP 1,174,438; EP 1,173,562; EP 1,170,008; EP 1,123,111; JP 2001/348340; U.S. Application No. 2002/103192; U.S. Pat. No. Application No. 2002/65282; U.S. Application No. 2002/61860; WO 02/51842; WO 02/50285; WO 02/46144; WO 02/46129; WO 02/30879; WO 02/26703; WO 02/26696; WO 01/70675; WO 01/42437; WO 01/38322; WO 01/18045; WO 01/14581; Furumai et al. (2002); Hinnebusch et al. (2002); Mai et al. (2002); Vigushin et al., (2002); Gottlicher et al. (2001); Jung (2001); Komatsu et al. (2001); Su et al., (2000).

IV. Methods of Treating Cardiac Hypertrophy

A. Therapeutic Regimens

Current medical management of cardiac hypertrophy in the setting of a cardiovascular disorder includes the use of at least two types of drugs: inhibitors of the rennin-angiotensoin system, and β-adrenergic blocking agents (Bristow, 1999). Therapeutic agents to treat pathologic hypertrophy in the setting of heart failure include angiotensin II converting enzyme (ACE) inhibitors and β-adrenergic receptor blocking agents (Eichhorn and Bristow, 1996). Other pharmaceutical agents that have been disclosed for treatment of cardiac hypertrophy include angiotensin II receptor antagonists (U.S. Pat. No. 5,604,251) and neuropeptide Y antagonists (WO 98/33791). Despite currently available pharmaceutical compounds, prevention and treatment of cardiac hypertrophy, and subsequent heart failure, continue to present a therapeutic challenge.

Non-pharmacological treatment is primarily used as an adjunct to pharmacological treatment. One means of non-pharmacological treatment involves reducing the sodium in the diet. In addition, non-pharmacological treatment also entails the elimination of certain precipitating drugs, including negative inotropic agents (e.g., certain calcium channel blockers and antiarrhythmic drugs like disopyramide), cardiotoxins (e.g., amphetamines), and plasma volume expanders (e.g., nonsteroidal anti-inflammatory agents and glucocorticoids).

In one embodiment of the present invention, methods for the treatment of cardiac hypertrophy or heart failure utilizing inhibitors of PKD are provided. For the purposes of the present application, treatment comprises reducing one or more of the symptoms of cardiac hypertrophy, such as reduced exercise capacity, reduced blood ejection volume, increased left ventricular end diastolic pressure, increased pulmonary capillary wedge pressure, reduced cardiac output, cardiac index, increased pulmonary artery pressures, increased left ventricular end systolic and diastolic dimensions, and increased left ventricular wall stress, wall tension and wall thickness-same for right ventricle. In addition, use of inhibitors of PKD may prevent cardiac hypertrophy and its associated symptoms from arising.

Treatment regimens would vary depending on the clinical situation. However, long term maintenance would appear to be appropriate in most circumstances. It also may be desirable treat hypertrophy with inhibitors of PKD intermittently, such as within brief window during disease progression.

B. Combined Therapy

In another embodiment, it is envisioned to use an inhibitor of PKD in combination with other therapeutic modalities. Thus, in addition to the therapies described above, one may also provide to the patient more “standard” pharmaceutical cardiac therapies. Examples of other therapies include, without limitation, so-called “beta blockers,” anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists and cytokine blockers/inhibitors, and HDAC inhibitors.

Combinations may be achieved by contacting cardiac cells with a single composition or pharmacological formulation that includes both agents, or by contacting the cell with two distinct compositions or formulations, at the same time, wherein one composition includes the expression construct and the other includes the agent. Alternatively, the therapy using an inhibitor of PKD may precede or follow administration of the other agent(s) by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either an inhibitor of PKD, or the other agent will be desired. In this regard, various combinations may be employed. By way of illustration, where the inhibitor of PKD is “A” and the other agent is “B”, the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated.

C. Pharmacological Therapeutic Agents

Pharmacological therapeutic agents and methods of administration, dosages, etc., are well known to those of skill in the art (see for example, the “Physicians Desk Reference”, Klaassen's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such invidual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

In addition, it should be noted that any of the following may be used to develop new sets of cardiac therapy target genes as β-blockers were used in the present examples (see below). While it is expected that many of these genes may overlap, new gene targets likely can be developed.
i. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present invention, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.
a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.
b. Resins/Bile Acid Sequesterants

Non-limiting examples of resins/bile acid sequesterants include cholestyramine (cholybar, questran), colestipol (colestid) and polidexide.
C. HMG CoA Reductase Inhibitors

Non-limiting examples of HMG CoA reductase inhibitors include lovastatin (mevacor), pravastatin (pravochol) or simvastatin (zocor).
d. Nicotinic Acid Derivatives

Non-limiting examples of nicotinic acid derivatives include nicotinate, acepimox, niceritrol, nicoclonate, nicomol and oxiniacic acid.
e. Thryroid Hormones and Analogs

Non-limiting examples of thyroid hormones and analogs thereof include etoroxate, thyropropic acid and thyroxine.
f. Miscellaneous Antihyperlipoproteinemics

Non-limiting examples of miscellaneous antihyperlipoproteinemics include acifran, azacosterol, benfluorex, β-benzalbutyramide, carnitine, chondroitin sulfate, clomestrone, detaxtran, dextran sulfate sodium, 5,8, 11, 14, 17-eicosapentaenoic acid, eritadenine, furazabol, meglutol, melinamide, mytatrienediol, omithine, γ-oryzanol, pantethine, pentaerythritol tetraacetate, α-phenylbutyramide, pirozadil, probucol (lorelco), β-sitosterol, sultosilic acid-piperazine salt, tiadenol, triparanol and xenbucin.
ii. Antiarteriosclerotics

Non-limiting examples of an antiarteriosclerotic include pyridinol carbamate.
iii. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.
a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.
b. Antiplatelet Agents

Non-limiting examples of antiplatelet agents include aspirin, a dextran, dipyridamole (persantin), heparin, sulfinpyranone (anturane) and ticlopidine (ticlid).
C. Thrombolytic Agents

Non-limiting examples of thrombolytic agents include tissue plaminogen activator (activase), plasmin, pro-urokinase, urokinase (abbokinase) streptokinase (streptase), anistreplase/APSAC (eminase).
iv. Blood Coagulants

In certain embodiments wherein a patient is suffering from a hemmorage or an increased likelyhood of hemmoraging, an agent that may enhance blood coagulation may be used. Non-limiting examples of a blood coagulation promoting agent include thrombolytic agent antagonists and anticoagulant antagonists.
a. Anticoagulant Antagonists

Non-limiting examples of anticoagulant antagonists include protamine and vitamine K1.
b. Thrombolytic Agent Antagonists and Antithrombotics

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.
V. Antiarrhythmic Agents

Non-limiting examples of antiarrhythmic agents include Class I antiarrythmic agents (sodium channel blockers), Class II antiarrythmic agents (beta-adrenergic blockers), Class II antiarrythmic agents (repolarization prolonging drugs), Class IV antiarrhythmic agents (calcium channel blockers) and miscellaneous antiarrythmic agents.
a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocaine), tocainide (tonocard) and mexiletine (mexitil). Non-limiting examples of Class IC antiarrhythmic agents include encainide (enkaid) and flecainide (tambocor).
b. Beta Blockers

Non-limiting examples of a beta blocker, otherwise known as a β-adrenergic blocker, a α-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.
C. Repolarization Prolonging Agents

Non-limiting examples of an agent that prolong repolarization, also known as a Class III antiarrhythmic agent, include amiodarone (cordarone) and sotalol (betapace).
d. Calcium Channel Blockers/Antagonist

Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexiline. In certain embodiments a calcium channel blocker comprises a long-acting dihydropyridine (nifedipine-type) calcium antagonist.
e. Miscellaneous Antiarrhythmic Agents

Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.
vi. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.
a. Alpha Blockers

Non-limiting examples of an alpha blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.
b. Alpha/Beta Blockers

In certain embodiments, an antihypertensive agent is both an alpha and beta adrenergic antagonist. Non-limiting examples of an alpha/beta blocker comprise labetalol (normodyne, trandate).
C. Anti-Angiotension II Agents

Non-limiting examples of anti-angiotension II agents include include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.
d. Sympatholytics

Non-limiting examples of a sympatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).
e. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(β-diethylaminoethyl ether), hexobendine, itramin tosylate, khellin, lidoflanine, mannitol hexanitrane, medibazine, nicorglycerin, pentaerythritol tetranitrate, pentrinitrol, perhexiline, pimethylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.
f. Miscellaneous Antihypertensives

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quantemary ammonium compound, a reserpine derivative or a suflonamide derivative.

Arylethanolamine Derivatives. Non-limiting examples of arylethanolamine derivatives include amosulalol, bufuralol, dilevalol, labetalol, pronethalol, sotalol and sulfinalol.

Benzothiadiazine Derivatives. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives. Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives. Non-limiting examples of dihydropyridine derivatives include amlodipine, felodipine, isradipine, nicardipine, nifedipine, nilvadipine, nisoldipine and nitrendipine.

Guanidine Derivatives. Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines. Non-limiting examples of hydrazines/phthalazines include budralazine, cadralazine, dihydralazine, endralazine, hydracarbazine, hydralazine, pheniprazine, pildralazine and todralazine.

Imidazole Derivatives. Non-limiting examples of imidazole derivatives include clonidine, lofexidine, phentolamine, tiamenidine and tolonidine.

Quanternary Ammonium Compounds. Non-limiting examples of quantemary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives. Non-limiting examples of reserpine derivatives include bietaserpine, deserpidine, rescinnamine, reserpine and syrosingopine.

Suflonamide Derivatives. Non-limiting examples of sulfonamide derivatives include ambuside, clopamide, furosemide, indapamide, quinethazone, tripamide and xipamide.
g. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.
vii. Treatment Agents for Congestive Heart Failure

Non-limiting examples of agents for the treatment of congestive heart failure include anti-angiotension II agents, afterload-preload reduction treatment, diuretics and inotropic agents.
a. Afterload-Preload Reduction

In certain embodiments, an animal patient that can not tolerate an angiotension antagonist may be treated with a combination therapy. Such therapy may combine adminstration of hydralazine (apresoline) and isosorbide dinitrate (isordil, sorbitrate).
b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexiline, ticrnafen and urea.
C. Inotropic Agents

Non-limiting examples of a positive inotropic agent, also known as a cardiotonic, include acefylline, an acetyldigitoxin, 2-amino-4-picoline, anrinone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include amrinone (inocor).
d. Antianginal Agents

Antianginal agents may comprise organonitrates, calcium channel blockers, beta blockers and combinations thereof.

Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

D. Surgical Therapeutic Agents

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

E. Drug Formulations and Routes for Administration to Patients

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector or cells, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

The active compositions of the present invention may include classic pharmaceutical preparations. Administration of these compositions according to the present invention may be via any common route so long as the target tissue is available via that route. This includes oral, nasal, or buccal. Alternatively, administration may be by intradermal, subcutaneous, intramuscular, intraperitoneal or intravenous injection, or by direct injection into cardiac tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

For oral administration the polypeptides of the present invention generally may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

V. Screening Methods

The present invention further comprises methods for identifying inhibitors of PKD that are useful in the prevention or treatment or reversal of cardiac hypertrophy or heart failure. These assays may comprise random screening of large libraries of candidate substances; alternatively, the assays may be used to focus on particular classes of compounds selected with an eye towards structural attributes that are believed to make them more likely to inhibit the function of PKD.

To identify an inhibitor of PKD, one generally will determine the function of a PKD in the presence and absence of the candidate substance. For example, a method generally comprises:
(a) providing a candidate modulator;
(b) admixing the candidate modulator with a PKD;
(c) measuring PKD kinase activity; and
(d) comparing the activity in step (c) with the activity in the absence of the candidate modulator,
wherein a difference between the measured activities indicates that the candidate modulator is, indeed, a modulator of the compound, cell or animal.

Assays also may be conducted in isolated cells, organs, or in living organisms. Typically, the kinase activity of PKD is measured by providing a class-II HDAC that is not phosphorylated and measuring the amount of label added by the PKD.

It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate substance” refers to any molecule that may potentially inhibit the kinase activity or cellular functions of PKD. The candidate substance may be a protein or fragment thereof, a small molecule, or even a nucleic acid. It may prove to be the case that the most useful pharmacological compounds will be compounds that are structurally related to known PKC inhibitors, listed elsewhere in this document. Using lead compounds to help develop improved compounds is known as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration, or which may affect the function of various other molecules. In one approach, one would generate a three-dimensional structure for a target molecule, or a fragment thereof. This could be accomplished by x-ray crystallography, computer modeling, or by a combination of both approaches.

It also is possible to use antibodies to ascertain the structure of a target compound, activator, or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecular libraries that are believed to meet the basic criteria for useful drugs in an effort to “brute force” the identification of useful compounds. Screening of such libraries, including combinatorially-generated libraries (e.g., peptide libraries), is a rapid and efficient way to screen large number of related (and unrelated) compounds for activity. Combinatorial approaches also lend themselves to rapid evolution of potential drugs by the creation of second, third, and fourth generation compounds modeled on active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

B. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

A technique for high throughput screening of compounds is described in WO 84/03564. Large numbers of small peptide test compounds are synthesized on a solid substrate, such as plastic pins or some other surface. Such peptides could be rapidly screening for their ability to bind and inhibit PKD.

C. In cyto Assays

The present invention also contemplates the screening of compounds for their ability to modulate PKD in cells. Various cell lines can be utilized for such screening assays, including cells specifically engineered for this purpose.

D. In vivo Assays

In vivo assays involve the use of various animal models of heart disease, including transgenic animals, that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for inhibitors may be conducted using an animal model derived from any of these species.

Treatment of animals with test compounds will involve the administration of the compound, in an appropriate form, to the animal. Administration will be by any route that could be utilized for clinical purposes. Determining the effectiveness of a compound in vivo may involve a variety of different criteria, including but not limited to. Also, measuring toxicity and dose response can be performed in animals in a more meaningful fashion than in in vitro or in cyto assays.

VI. Purification of Proteins

It will be desirable to purify PKD. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

VII. Vectors for Cloning, Gene Transfer and Expression

Within certain embodiments expression vectors are employed to express a PKD polypeptide product, which can then be purified. In other embodiments, the expression vectors may be used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, the native PKD promoter will be employed to drive expression of either the corresponding PKD gene, a heterologous PKD gene, a screenable or selectable marker gene, or any other gene of interest.

In other embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 1 and 2 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 1 and Table 2). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 1
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler |
| | et al., 1987; Weinberger et al., 1984; Kiledjian |
| | et al., 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo |
| | et al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson |
| | et al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; |
| | Imbra et al., 1986; Kadesch et al., 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi |
| | et al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 2
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa |
| | | et al., 1987, Karin et al., |
| | | 1987; Angel et al., 1987b; |
| | | McNeall et al., 1989 |
| MMTV | Glucocorticoids | Huang et al., 1981; Lee |
| (mouse | | et al., 1981; Majors et al., |
| mammary | | 1983; Chandler et al., |
| tumor virus) | | 1983; Ponta et al., 1985; |
| | | Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class | Interferon | Blanar et al., 1989 |
| I Gene H-2κb | | |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor | | |
| Thyroid Stimulating | Thyroid Hormone | Chatterjee et al., 1989 |
| Hormone α Gene | | |

Of particular interest are muscle specific promoters, and more particularly, cardiac specific promoters. These include the myosin light chain-2 promoter (Franz et al., 1994; Kelly et al., 1995), the alpha actin promoter (Moss et al., 1996), the troponin 1 promoter (Bhavsar et al., 1996); the Na⁺/Ca²⁺ exchanger promoter (Barnes et al., 1997), the dystrophin promoter (Kimura et al., 1997), the alpha7 integrin promoter (Ziober and Kramer, 1996), the brain natriuretic peptide promoter (LaPointe et al., 1996) and the alpha B-crystallin/small heat shock protein promoter (Gopal-Srivastava, 1995), alpha myosin heavy chain promoter (Yamauchi-Takihara et al., 1989) and the ANF promoter (LaPointe et al., 1988).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

B. Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

C. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

D. Delivery of Expression Vectors

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by AdS DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al., introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

VIII. Methods of Making Transgenic Mice

A particular embodiment of the present invention provides transgenic animals that express a heterologous PKD gene under the control of a promoter. Transgenic animals expressing a PKD encoding nucleic acid under the control of an inducible or a constitutive promoter, recombinant cell lines derived from such animals, and transgenic embryos may be useful in determining the exact role that PKD plays in the development and differentiation of cardiomyocytes and in the development of pathologic cardiac hypertrophy and heart failure. Furthermore, these transgenic animals may provide an insight into heart development. The use of constitutively expressed PKD encoding nucleic acid provides a model for over- or unregulated expression. Also, transgenic animals which are “knocked out” for PKD, in one or both alleles are contemplated.

In a general aspect, a transgenic animal is produced by the integration of a given transgene into the genome in a manner that permits the expression of the transgene. Methods for producing transgenic animals are generally described by Wagner and Hoppe (U.S. Pat. No. 4,873,191; which is incorporated herein by reference), and Brinster et al., 1985; which is incorporated herein by reference in its entirety).

Typically, a gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish.

DNA clones for microinjection can be prepared by any means known in the art. For example, DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris,pH 7.4, and 1 mM EDTA) and purified on an Elutip-D™ column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer. For microinjection, DNA concentrations are adjusted to 3 μg/ml in 5 mM Tris, pH 7.4 and 0.1 mM EDTA. Other methods for purification of DNA for microinjection are described in in Palmiter et al. (1982); and in Sambrook et al. (2001).

In an exemplary microinjection procedure, female mice six weeks of age are induced to superovulate with a 5 IU injection (0.1 cc, ip) of pregnant mare serum gonadotropin (PMSG; Sigma) followed 48 hours later by a 5 IU injection (0.1 cc, ip) of human chorionic gonadotropin (hCG; Sigma). Females are placed with males immediately after hCG injection. Twenty-one hours after hCG injection, the mated females are sacrificed by CO2 asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO₂, 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

IX. Antibodies Reactive With PKD

In another aspect, the present invention contemplates an antibody that is immunoreactive with a PKD molecule of the present invention, or any portion thereof. An antibody can be a polyclonal or a monoclonal antibody. In a preferred embodiment, an antibody is a monoclonal antibody. Means for preparing and characterizing antibodies are well known in the art (see, e.g., Harlow and Lane, 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

It is proposed that the monoclonal antibodies of the present invention will find useful application in standard immunochemical procedures, such as ELISA and Western blot methods and in immunohistochemical procedures such as tissue staining, as well as in other procedures which may utilize antibodies specific to PKD-related antigen epitopes.

In general, both polyclonal, monoclonal, and single-chain antibodies against PKD may be used in a variety of embodiments. A particularly useful application of such antibodies is in purifying native or recombinant PKD, for example, using an antibody affinity column. The operation of all accepted immunological techniques will be known to those of skill in the art in light of the present disclosure.

Means for preparing and characterizing antibodies are well known in the art (see, e.g., Harlow and Lane, 1988; incorporated herein by reference). More specific examples of monoclonal antibody preparation are given in the examples below.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified PKD protein, polypeptide or peptide or cell expressing high levels of PKD. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

X. Definitions

As used herein, the term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. Though the precise physiological mechanisms of heart failure are not entirely understood, heart failure is generally believed to involve disorders in several cardiac autonomic properties, including sympathetic, parasympathetic, and baroreceptor responses. The phrase “manifestations of heart failure” is used broadly to encompass all of the sequelae associated with heart failure, such as shortness of breath, pitting edema, an enlarged tender liver, engorged neck veins, pulmonary rales and the like including laboratory findings associated with heart failure.

The term “treatment” or grammatical equivalents encompasses the improvement and/or reversal of the symptoms of heart failure (i.e., the ability of the heart to pump blood). “Improvement in the physiologic function” of the heart may be assessed using any of the measurements described herein (e.g., measurement of ejection fraction, fractional shortening, left ventricular internal dimension, heart rate, etc.), as well as any effect upon the animal's survival. In use of animal models, the response of treated transgenic animals and untreated transgenic animals is compared using any of the assays described herein (in addition, treated and untreated non-transgenic animals may be included as controls). A compound which causes an improvement in any parameter associated with heart failure used in the screening methods of the instant invention may thereby be identified as a therapeutic compound.

The term “dilated cardiomyopathy” refers to a type of heart failure characterized by the presence of a symmetrically dilated left ventricle with poor systolic contractile function and, in addition, frequently involves the right ventricle.

The term “compound” refers to any chemical entity, pharmaceutical, drug, and the like that can be used to treat or prevent a disease, illness, sickness, or disorder of bodily function. Compounds comprise both known and potential therapeutic compounds. A compound can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of heart failure.

As used herein, the term “agonist” refers to molecules or compounds which mimic the action of a “native” or “natural” compound. Agonists may be homologous to these natural compounds in respect to conformation, charge or other characteristics. Thus, agonists may be recognized by receptors expressed on cell surfaces. This recognition may result in physiologic and/or biochemical changes within the cell, such that the cell reacts to the presence of the agonist in the same manner as if the natural compound was present. Agonists may include proteins, nucleic acids, carbohydrates, or any other molecules that interact with a molecule, receptor, and/or pathway of interest.

As used herein, the term “cardiac hypertrophy” refers to the process in which adult cardiac myocytes respond to stress through hypertrophic growth. Such growth is characterized by cell size increases without cell division, assembling of additional sarcomeres within the cell to maximize force generation, and an activation of a fetal cardiac gene program. Cardiac hypertrophy is often associated with increased risk of morbidity and mortality, and thus studies aimed at understanding the molecular mechanisms of cardiac hypertrophy could have a significant impact on human health.

As used herein, the terms “antagonist” and “inhibitor” refer to molecules, compounds, or nucleic acids which inhibit the action of a cellular factor that may be involved in cardiac hypertrophy. Antagonists may or may not be homologous to these natural compounds in respect to conformation, charge or other characteristics. Thus, antagonists may be recognized by the same or different receptors that are recognized by an agonist. Antagonists may have allosteric effects which prevent the action of an agonist. Alternatively, antagonists may prevent the function of the agonist. In contrast to the agonists, antagonistic compounds do not result in pathologic and/or biochemical changes within the cell such that the cell reacts to the presence of the antagonist in the same manner as if the cellular factor was present. Antagonists and inhibitors may include proteins, nucleic acids, carbohydrates, or any other molecules which bind or interact with a receptor, molecule, and/or pathway of interest.

As used herein, the term “modulate” refers to a change or an alteration in a biological activity. Modulation may be an increase or a decrease in protein activity, a change in kinase activity, a change in binding characteristics, or any other change in the biological, functional, or immunological properties associated with the activity of a protein or other structure of interest. The term “modulator” refers to any molecule or compound which is capable of changing or altering biological activity as described above.

The term “β-adrenergic receptor antagonist” refers to a chemical compound or entity that is capable of blocking, either partially or completely, the beta (β) type of adrenoreceptors (i.e., receptors of the adrenergic system that respond to catecholamines, especially norepinephrine). Some β-adrenergic receptor antagonists exhibit a degree of specificity for one receptor sybtype (generally β₁); such antagonists are termed “β₁-specific adrenergic receptor antagonists” and “β₂-specific adrenergic receptor antagonists.” The term β-adrenergic receptor antagonist” refers to chemical compounds that are selective and non-selective antagonists. Examples of β-adrenergic receptor antagonists include, but are not limited to, acebutolol, atenolol, butoxamine, carteolol, esmolol, labetolol, metoprolol, nadolol, penbutolol, propanolol, and timolol. The use of derivatives of known β-adrenergic receptor antagonists is encompassed by the methods of the present invention. Indeed any compound, which functionally behaves as a β-adrenergic receptor antagonist is encompassed by the methods of the present invention.

The terms “angiotensin-converting enzyme inhibitor” or “ACE inhibitor” refer to a chemical compound or entity that is capable of inhibiting, either partially or completely, the enzyme involved in the conversion of the relatively inactive angiotensin I to the active angiotensin II in the rennin-angiotensin system. In addition, the ACE inhibitors concomitantly inhibit the degradation of bradykinin, which likely significantly enhances the antihypertensive effect of the ACE inhibitors. Examples of ACE inhibitors include, but are not limited to, benazepril, captopril, enalopril, fosinopril, lisinopril, quiapril and ramipril. The use of derivatives of known ACE inhibitors is encompassed by the methods of the present invention. Indeed any compound, which functionally behaves as an ACE inhibitor, is encompassed by the methods of the present invention.

As used herein, the term “genotypes” refers to the actual genetic make-up of an organism, while “phenotype” refers to physical traits displayed by an individual. In addition, the “phenotype” is the result of selective expression of the genome (i.e., it is an expression of the cell history and its response to the extracellular environment). Indeed, the human genome contains an estimated 30,000-35,000 genes. In each cell type, only a small (i.e., 10-15%) fraction of these genes are expressed.

### XI. EXAMPLES

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Chemical reagents and plasmids. Phorbol 12-myristate 13-acetate (PMA), 8-Br-cAMP, pCPT-cGMP, and anisomycin were obtained from Sigma Chemical (St. Louis, Mo.). The following kinase inhibitors were purchased from the indicated vendors: bisindolylmaleimide I and Gö6976 (A.G. Scientific, San Diego, Calif.), KN93, SB216763 and wortmannin (BIOMOL, Plymouth Meeting, Pa.), Gö6983, staurosporine, PD98059, wortmannin, U1026, Y-27632, Rapamycin and DAG Kinase Inhibitor II (Calbiochem). KN93, wortmannin and staurosporine were used at 1 mM. U1026, HA1077, Y-27632, DAG Kinase inhibitor II, SB216763 and Bis I were used at 10 mM. Rapamycin was employed at 30 ng/ml. Phenylephrine and endothelin-1 were purchased from Sigma. Mammalian expression vectors encoding PKD isoforms were kindly provided by Alex Toker and have been described elsewhere (Storz and Toker, 2003).

Cell culture and transfection assays. COS cells were maintained in DMEM with FBS (10%), L-glutamine (2 mM), and penicillin-streptomycin. Transfection of COS cells was performed with Fugene 6 (Roche Molecular Biochemicals) according to manufacturer's instructions. For HDAC localization experiments, cells were treated 16-24 hours after transfection with PMA (100 nM), ionomycin (1 mM), 8-Br-cAMP (1 mM), pCPT-GMP (1 mM), or anisomycin (1 mM). Where noted, specific protein kinase inhibitors were added 30 min prior to the addition of any chemical stimulus. GFP-HDAC5 was visualized with standard fluorescent microscopic techniques. For indirect immunofluorescence of FLAG-HDAC5, COS cells were seeded on glass coverslips, transfected, and treated as above. After specific treatment, cells were fixed with buffered formalin (10%) and stained in PBS containing BSA (3%) and Nonidet P-40 (0.1%). Flag M2 antibody (Sigma) was used at a concentration of 1:200. Secondary fluoresceine-conjugated antibody (Vector Laboratories) was also used at a concentration of 1:200. Staining of cardiomyocytes for sarcomeres and atrial natriuretic factor (ANF) was performed by indirect immunofluorescence detection as above with antibodies directed against sarcomeric α-actinin (Sigma) and ANF (Peninsula Laboratories), respectively.

Cardiac myocyte culture and adenoviral infection. Neonatal rat cardiac myocytes (NRVM) were isolated from 1-2 day Sprague Dawley rats as previously described (Antos et al., 2003). For adenovirus production, cDNAs encoding LacZ or FLAG-tagged HDAC5 (S259/498A) were subcloned into the pACCMV vector and co-transfected with pJM17 into 293 cells. Primary lysates were used to re-infect 293 cells and viral plaques were obtained using the agar overlay method. Complementary DNA for full-length human HDAC5 (encoding 1122 amino acids) was fused to sequences encoding enhanced green fluorescent protein (EGFP; Clontech) in pcDNA3.1+(Invitrogen). The resultant construct encodes GFP fused in-frame to the amino-terminus of HDAC5. A construct encoding GFP fused to HDAC5 containing alanines in place of serines 259 and 498 was generated in the same manner. GFP-HDAC5 cDNAs were subcloned into pACCMV for adenovirus production. Clonal populations of adenoviruses were amplified and titered.

Co-immunoprecipitation assays. Flag-HDAC5 expression plasmid was transfected into COS cells treated as described above. Treated cells were harvested in Tris (50 mM, pH 7.4), NaCl (150 mM), EDTA (1 mM), and Triton X-100 (1%). Cells were further disrupted by passage through a 22-guage needle and cell debris removed by centrifugation. Flag-HDAC5 was immunoprecipitated with M2-agarose conjugate (Sigma) and thoroughly washed. Bound proteins were resolved by SDS-PAGE and western blot analysis performed using Flag M2 (Sigma) or 14-3-3 antibody (Santa Cruz Biotechnology). For studies with NRVM, whole-cell proteins extracts were prepared from cells expressing GFP-HDAC5 using the same buffer supplemented with protease inhibitor cocktail (Complete; Roche), PMSF (1 mM) and phosphatase inhibitors [sodium pyrophosphate (1 mM), sodium fluoride (2 mM), b-glycerol phosphate (10 mM), sodium molybdate (1 mM), sodium orthovanadate (1 mM)]. Lysates were sonicated briefly and clarified by centrifugation. For immunoprecipitation, protein lysates were exposed to HDAC5-specific antiserum (19) and protein G sepharose beads (Amersham Biosciences). Immunoprecipitates were washed 5 times with lysis buffer, resolved by SDS-PAGE and immunoblotted with mouse monoclonal antibodies specific for either GFP (BD Biosciences; 1:2,500 dilution) or 14-3-3 (Santa Cruz [H-8]; 1:1000 dilution). Associated PKD was detected by immunoblotting with rabbit polyclonal antibodies against either PKD-1, PKD-1 phosphorylated at serines 744 and 748, or PKD-1 phosphorylated at serine 916 (Cell Signaling Technologies) were employed at 1:1000 dilutions.

In vitro kinase assays. Flag-HDAC5 was immunoprecipitated with anti-Flag M2 antibody, as described above. Bound Flag-HDAC5 was washed and equilibrated with kinase buffer (Tris (25 mM, pH 7.4), MgCl2 (10 mM), DTT (1 mM). Following equilibration, kinase reaction mix was added (Kinase buffer plus ATP (0.1 mM) and 50 mCi [g-32P]-ATP). Kinase reactions were carried out at 30° C. for 30 minutes and terminated by the addition of an equal volume of 2×SDS-PAGE loading buffer. Phosphorylated proteins were resolved by SDS-PAGE and visualized by autoradiography.

GFP-HDAC5 localization studies. For analysis of GFP-HDAC5 in NRVM, cells were plated in the presence of adenovirus (multiplicity of infection=˜50-100) on gelatin-coated 96-well dishes (Costar; 1×104 cells/well) in DMEM containing fetal bovine serum (FBS) (10%), L-glutamine (2 mM), and penicillin-streptomycin. After overnight culture, cells were washed with serum-free medium and maintained in DMEM (100 ml) supplemented with Neutridoma-SP (0.1%; Roche Applied Science), which contains albumin, insulin, transferrin, and other defined organic and inorganic compounds. Following culture in serum-free medium (3 hrs), cells were exposed to kinase inhibitors (30 min) prior to stimulation with agonist for 2.5 hrs. Cells were washed with PBS and fixed with 10% formalin in PBS containing Hoechst dye 33342 (H-3570, Molecular Probes). Images were captured at 40× magnification using a fluorescence microscope (Nikon Eclipse TS 100) equipped with a digital camera (Photometrics CoolSNAP HQ) and MetaMorph imaging software. Relative abundance of GFP-HDAC5 in the nucleus versus the cytoplasm was quantified employing the High Content Imaging System (Cellomics, Inc., Pittsburgh, Pa.), which demarcates nuclei based on Hoechst fluorescence and defines a cytoplasmic ring based on these nuclear dimensions. Values for HDAC5 localization represent averages from a minimum of 200 cells per experimental condition.

RNA analysis. NRVM were plated on gelatin-coated 10 cm dishes (2×10⁶ cells/dish). Following the indicated treatments, RNA was isolated from cardiomyocytes using Trizol Reagent (Gibco/BRL). Total RNA (2 μg) was vacuum blotted onto nitrocellulose membranes (Bio-Rad) using a 96-well format dot blotter (Bio-Rad). Membranes were blocked in 4×SSC containing SDS (1%), 5× Denhardt's Reagent, sodium pyrophosphate (0.05%), and 100 μg/ml sonicated salmon sperm DNA (4 hrs at 500° C.) and incubated with 32P-end-labeled oligonucleotide probes (1×10⁶ cpm/ml; 14 hrs at 500° C.). Sequences of oligonucleotides were as follows: ANF, 5′-aatgtgaccaagctgcgtgacacaccacaagggcttaggatcttttgcgatctgctcaag-3′; BNP, 5′-tgaactatgtgccatcttggaatttcgaagtctctcct-3′; α-SK-actin 5′-tggagcaaaacagaatggctggctttaatgcttcaagttttccatttcctttccacaggg-3′; GAPDH 5′-ggaacatgtagaccatgtagttgaggtcaatgaag-3′). Blots were washed twice with 0.5×SSC containing SDS (0.1%; 10 minutes at 500° C.) and analyzed by autoradiography.

Mammalian two-hybrid analysis. A mammalian expression vector encoding the GAL4 DNA binding domain fused to the amino-terminus of human HDAC5 (amino acids 2-664) was generated in the pMl expression vector (Sadowski). GAL4-HDAC5 fusions harboring alanine in place of either serine 259 and/or 498 were constructed in an analogous manner. A construct encoding the herpesvirus VP16 transcriptional activation domain fused to the amino terminus of 14-3-3 sigma was generated employing pVP16 (Clontech). COS cells were transiently transfected with vectors for GAL4-HDAC5, VP16-14-3-3 and a luciferase reporter gene under the control of five copies of a GAL4 DNA binding site (5XUAS-luciferase) in the absence or presence of a construct for constitutively active PKD-1. Forty-eight hrs post-transfection, cells were harvested and luciferase levels quantified employing the Luciferase Assay Kit (Promega).

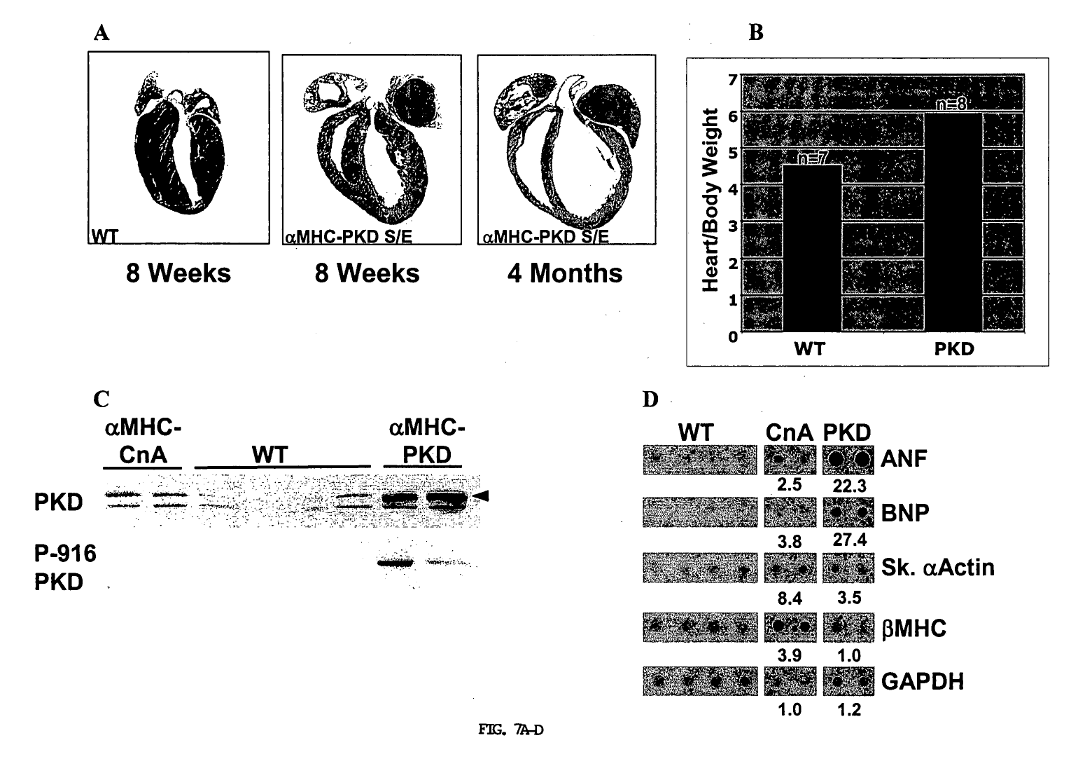

Transgenic mouse production. A cDNA encoding a constitutively active form of PKD was cloned downstream of the cardiac-specific α-myosin heavy chain promoter. This vector was injected into B6C3F1 mouse oocytes and implanted into surrogate female ICR mice. Transgenic offspring were identified by PCR with transgene-specific primers. Results are displayed in FIGS. 7A-D.

### Example 2

### Results

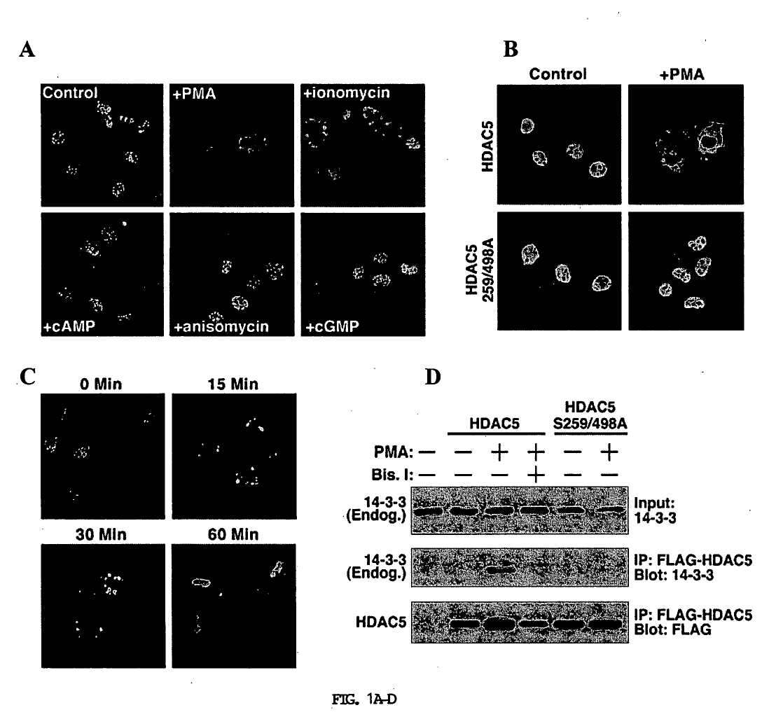

A PKC-dependent pathway stimulates nuclear export of HDAC5. To further define the signaling pathways leading to phosphorylation and nuclear export of class II HDACs, the inventors tested a variety of activators of protein kinase pathways for their ability to stimulate nuclear export of HDAC5 in COS cells. HDAC5 is primarily located in the nucleus of COS cells allowing for a convenient system to assess nuclear export. Activators of PKA (8-Br-cAMP), PKG (pCPT-GMP), PKC (PMA), CaMK (ionomycin), and Jun-N-terminal kinase (anisomycin) were tested for their ability to activate nuclear export of HDAC5 fused to GFP. Among these compounds, only ionomycin and PMA stimulated nuclear export of GFP-HDAC5 (FIG. 1A). PMA was a more potent stimulator of export than ionomycin at the concentrations tested.

Nuclear export of HDAC5 and other class II HDACs in response to CaMK signaling requires two serines located in the N-terminal regions of the HDAC proteins (Grozinger and Schreiber, 2000; McKinsey et al., 2000). An HDAC5 mutant (HDAC5-S/A) containing alanine substitutions of these serine residues (residues 249 and 498) was not exported in response to PMA treatment (FIG. 1B), verifying the requisite role of these sites for responsiveness to PKC activation. Nuclear export of HDAC5 was initiated within 15 min following addition of PMA and was complete by 30 min (FIG. 1C).

Phosphorylation of serines 249 and 498 in HDAC5 creates docking sites for 14-3-3 proteins, which escort HDAC5 to the cytoplasm (Grozinger and Schreiber, 2000; McKinsey et al., 2000). To further confirm that PMA promoted phosphorylation of these sites, the inventors analyzed the interaction of HDAC5 with 14-3-3 in co-immunoprecipitation assays. As shown in FIG. 1D, the association of 14-3-3 with HDAC5 was enhanced in the presence of PMA. In contrast, the HDAC5-S/A mutant failed to respond to PMA and did not associate with 14-3-3. The inventors therefore conclude that PKC signaling leads to the phosphorylation of serines 249 and 498 of HDAC5 and consequent nuclear export through a 14-3-3-dependent mechanism.

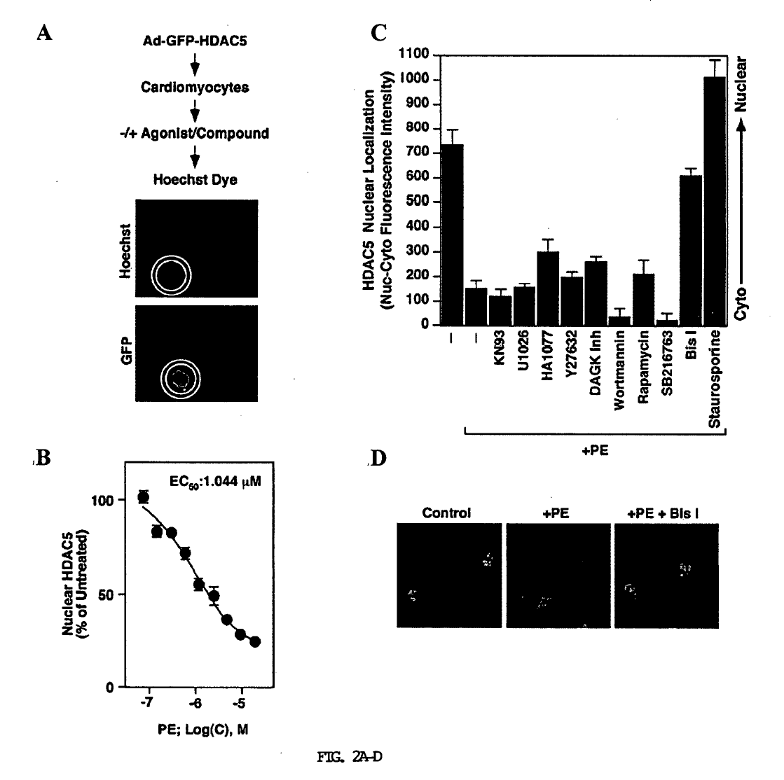

PKC-dependent nuclear export of HDAC5 in cardiac myocytes. To begin to address the role of PKC signaling in control of HDAC5 trafficking during cardiac hypertrophy, the inventors developed a quantitative assay to measure agonist-dependent nuclear export of HDAC5 in primary cardiomyocytes. This assay employs the Cellomics High Content Imaging System, which rapidly quantifies nuclear and cytoplasmic GFP fluorescence intensity and provides a read-out of the difference in intensity between the two subcellular compartments (FIG. 2A). To validate the assay, rat neonatal ventricular cardiac myocytes (NRVMs) were infected with an adenovirus expressing GFP-HDAC5 (Ad-GFP-HDAC5) and stimulated with increasing doses of the α-1-adrenergic agonist phenylephrine (PE), a hypertrophic agonist that promotes nuclear export of HDAC5 (Bush et al., 2004). As shown in FIG. 2B, PE triggered nuclear export of HDAC5 in a concentration-dependent manner. These results were confirmed by visual inspection of the cells (data not shown).

Having established the validity of the quantitative nuclear export assay, the inventors next tested an array of inhibitors of different kinases for their abilities to block PE-induced translocation of HDAC5 out of the nucleus. The general serine/threonine protein kinase inhibitor staurosporine and the PKC inhibitor bisindolylmaleimide I (Bis I) were effective in blocking PE-dependent export of HDAC5 (FIGS. 2C and 2D). In contrast, inhibitors of CaMK (KN93), MEK1 (U1026), ROCK (Y-27632), diacylglycerol kinase (DAGK inhibitor II), PI3-kinase (wortmannin), S6 kinase (rapamycin), GSK (SB216763) or an inhibitor of PKG, MLCK and PKA (HA1077), did not significantly affect PE-induced nuclear export of HDAC5.

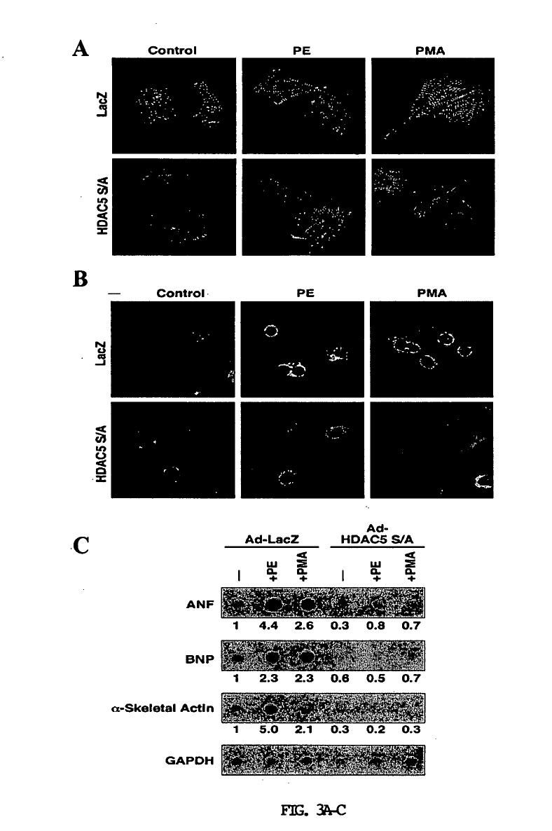

PKC signaling induces cardiac hypertrophy via HDAC phosphorylation. PKC activation has been shown to be sufficient, and in some cases necessary, for cardiomyocyte hypertrophy (see Antos, 2003; Dunnmon et al., 1990). The above results implicate PKC-dependent nuclear export of HDAC5 or other class II HDACs in the development of cardiomyocyte hypertrophy. To address this possibility, the inventors examined whether hypertrophy in response to PKC activation required phosphorylation and nuclear export of class II HDACs. NRVMs were infected with adenoviruses encoding the signal-resistant HDAC5-S/A mutant protein or lacZ as a control. As shown in FIG. 3A, expression of the HDAC5-S/A mutant in primary cardiomyocytes prevented sarcomere assembly and cell enlargement in response to PE or PMA.

Cardiac hypertrophy is associated with reactivation of a pathological “fetal” gene program, which includes the genes encoding atrial natriuretic factor (ANF), brain natriuretic factor (BNP) and α-skeletal actin. Agonist-dependent elevation of ANF expression can also be examined by immunostaining cardiomyocytes with ANF-specific antibodies. As shown in FIG. 3B, prominent perinuclear ANF protein expression was observed in NRVMs treated with PE or PMA. Agonist-dependent induction of ANF expression was unaffected by ectopic expression of LacZ, but was markedly reduced in the presence of signal-resistant HDAC5. In addition, non-phosphorylatable HDAC5 blocked PE- and PMA-mediated induction of ANF transcripts, as well as those for BNP and α-skeletal actin. Together, these results indicate that PKC signaling triggers cardiac hypertrophy in part by stimulating nuclear export of class II HDACs.

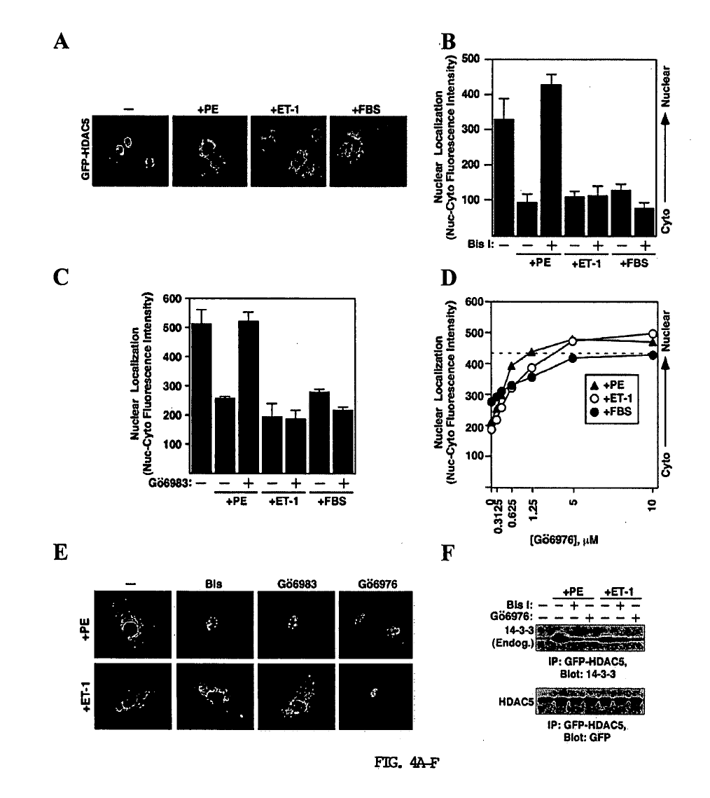

Differential sensitivity of HDAC5 nuclear export to PKC inhibition. The inventors next examined whether nuclear export of HDAC5 in response to other hypertrophic signals was also dependent on PKC signaling. Endothelin-1 (ET-1) and fetal bovine serum (FBS), which stimulate hypertrophy, also effectively promote nuclear export of HDAC5 (data unpublished). However, in contrast to its inhibitory effect on PE-dependent HDAC5 nuclear export, Bis I had no effect on the nuclear export of HDAC5 in response to ET-1 or FBS (FIG. 4A). These findings suggested that PE triggers different kinase pathways than ET-1 and FBS to promote nuclear export of HDAC5.

To further examine the nature of the protein kinase effectors of the above hypertrophic agonists, the inventors tested additional PKC inhibitors for their possible effects on HDAC5 nuclear export. The activity of Go6983, another general inhibitor of PKCs, paralleled that of Bis I, inhibiting HDAC5 nuclear export in response to PE, but not ET-1 or FBS (FIGS. 4B and 4D). In contrast, Gö6976, a specific inhibitor of the calcium-dependent PKCa and b isozymes, efficiently blocked nuclear export of HDAC5 triggered by PE, ET-1 or FBS (FIGS. 4C and D). The differential effects of the above inhibitors on HDAC5 nuclear export were paralleled by their effects on association of 14-3-3 with HDAC5, an indicator of HDAC5 phosphorylation. Gö6976, but not Bis I, blocked association of HDAC5 and 14-3-3 in response to both PE and ET-1 (FIG. 4E).

The ability of Go6976, but not Bis I or Go6983, to block HDAC5 nuclear export in response to multiple agonists was seemingly paradoxical, since the latter compounds block PKCa and b as effectively as Gö6976. However, this inhibitor profile was similar to that used by others to distinguish the actions of PKCa or b from PKD/PKCm (Zugaza et al., 1996), which is sensitive to Gö6976 but not to Bis I or Gö6983 (Gschwendt et al., 1996).

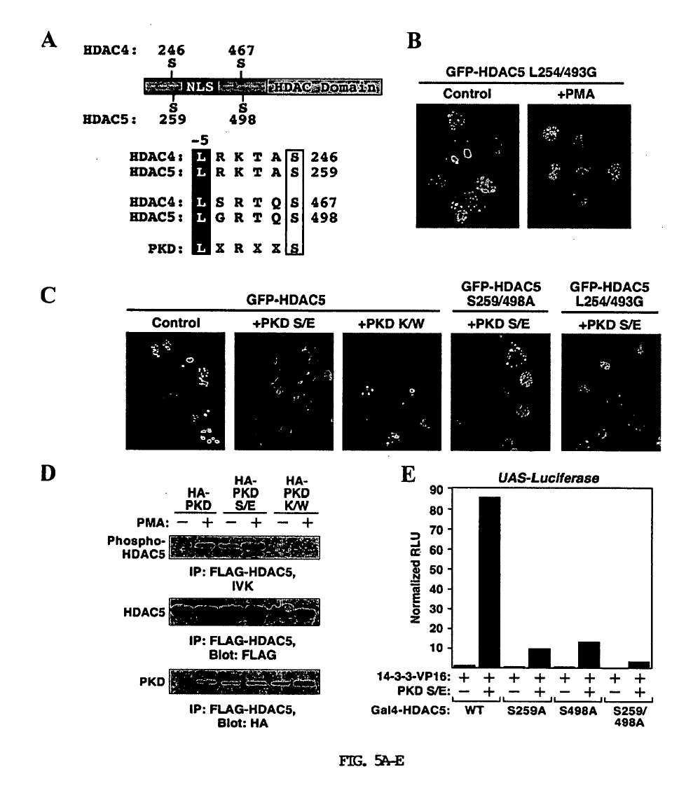

Protein Kinase D stimulates nuclear export of HDAC5. In light of the above results, which suggested the possible involvement of PKD in agonist-dependent nuclear export of HDAC5, the inventors examined the amino acid sequence surrounding the signal responsive serines in HDAC5 for a potential PKD consensus phosphorylation site. PKD has a strong preference for a leucine residue at the −5 position relative to the phosphorylated serine (Nishikawa et al., 1997). HDAC5 contains a leucine at this position relative to both signal-responsive serine residues (FIG. 5A). Interestingly, the class II HDACs 4, 7 and 9 also contain leucine at this position.

To assess the importance of the leucine at position −5, the inventors mutated leucines 254 and 493 in HDAC5 to glycines, leaving the actual phosphorylation sites intact. This HDAC5 mutant (L254/493G) was constitutively localized to the nucleus and was completely refractory to PMA (FIG. 5B). Further support for the involvement of PKD in HDAC5 nuclear export was provided by transfection assays in which an activated form of PKD (PKD S/E), but not a catalytically inactive mutant (PKD K/W), effectively stimulated nuclear export of HDAC5 (FIG. 5C). Mutation of the signal-responsive serine residues or the leucines at positions 254 and 493 of HDAC5 abolished nuclear export in response to PKD (FIG. 5C).

To further explore the potential role of PKD as an HDAC5 nuclear export kinase, the inventors performed co-immunoprecipitation and in vitro kinase assays. Co-immunoprecipitation of HDAC5 and PKD followed by an in vitro kinase assay confirmed that PKD directly phosphorylated HDAC5. As shown in FIG. 5D, co-transfection of PKD resulted in little phosphorylation of HDAC5. Treatment of the cells with PMA increased the degree of HDAC5 phosphorylation coincident with PKD binding. Binding and phosphorylation of HDAC5 by activated PKD S/E did not require PMA although PMA treatment enhanced phosphorylation of HDAC5, perhaps owing to the presence of endogenous PKD in immune complexes. No phosphorlyation of HDAC5 was observed with the catalytically inactive mutant PKD K/W. Interestingly, however, PKD K/W bound to HDAC5 even in the absence of PMA.

PKD also increased the interaction between HDAC5 and 14-3-3 as assessed by a mammalian two-hybrid assay in which HDAC5 was fused to the GAL4 DNA binding domain and 14-3-3 to the VP16 transcription activation domain (FIG. 5E). Mutation of either signal responsive serine in HDAC5 markedly decreased the interaction between HDAC5 and 14-3-3, and mutation of both signal responsive serines completely abolished binding of HDAC5 to 14-3-3.

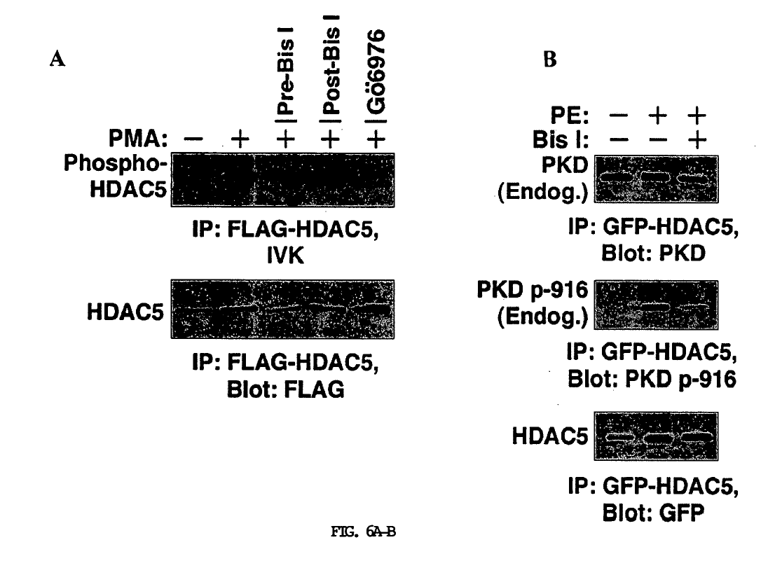

PKD is a cardiac HDAC5 kinase. The inventors next examined whether PKD could serve as an HDAC kinase in cardiomyocytes. Cells were infected with adenovirus encoding Flag-HDAC5 and treated with PMA. Increased HDAC5 phosphorylation was observed in an in vitro kinase assay performed with FLAG-HDAC5 immunoprecipitated from PMA-treated cells (FIG. 6A). Incubation of the cells with Bis I before addition of PMA blocked phosphorylation of HDAC5. However, addition of Bis I directly to the kinase reaction had no effect while Gö6976 blocked phosphorylation of HDAC5. These results suggest PKD is capable of binding HDAC5 in cardiac myocytes and that Bis I blocks the PMA-induced activation of the kinase, while Gö6976 is able to directly inhibit HDAC5-bound PKD.

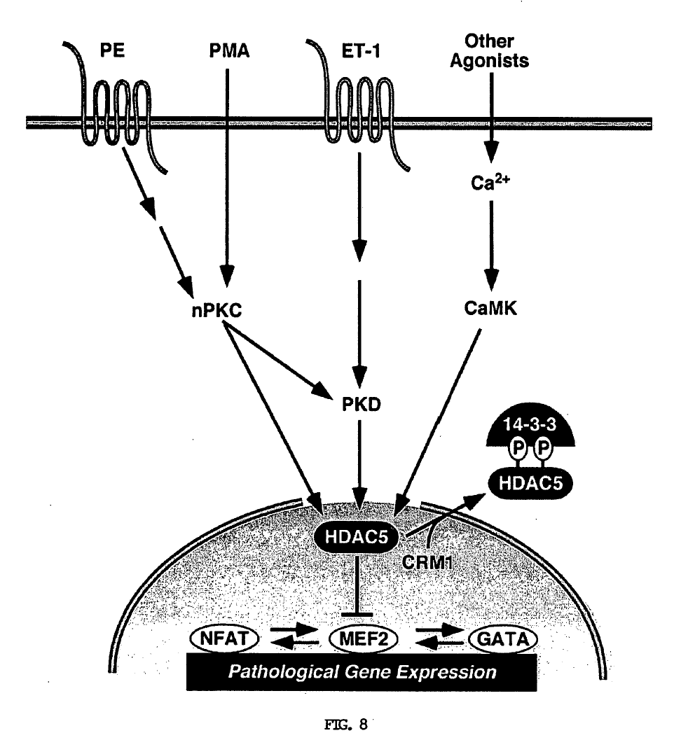

The ability of PKD to interact with HDAC5 in cardiac myocytes was further addressed by sequential immunoprecipitation and immunoblotting. NRVMs were infected with GFP-HDAC5 encoding adenovirus and treated with PE in the absence or presence of Bis. I. As shown in FIG. 6A, endogenous PKD efficiently co-immunoprecipitated with HDAC5. PKD was associated with HDAC5 in the absence of agonist and became activated in a PKC-dependent manner following PE treatment. The results further suggest that PKD is a cardiac class II HDAC kinase and support the model proposed in FIG. 8.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### XII. References

The following references, to the extent that they provide exemplary procedural or other details supplementary to those set forth herein, are specifically incorporated herein by reference.
U.S. App. 2002/103192
U.S. App. 2002/65282
U.S. App. 2002/61860
U.S. Pat. No. 4,196,265
U.S. Pat. No. 4,415,723
U.S. Pat. No. 4,458,066
U.S. Pat. No. 4,873,191
U.S. Pat. No. 5,354,855
U.S. Pat. No. 5,604,251
U.S. Pat. No. 5,795,715
U.S. Pat. No. 5,889,136
U.S. Pat. No. 5,955,501
U.S. Pat. No. 6,043,270
U.S. Pat. No. 6,080,784
U.S. Pat. No. 6,441,020
U.S. Pat. No. 6,528,294
Angel et al., Mol. Cell. Biol., 7:2256, 1987.
Angel et al., Mol. Cell. Biol., 7:2256, 1987a.
Angel et al., Cell, 49:729, 1987b.
Antos, et al., 2003. J. Biol. Chem., 278:28930-28937, 2003.
Atchison and Perry, Cell, 46:253, 1986.
Atchison and Perry, Cell, 48:121, 1987.
AU 6,794,700
AU 9,013,101
AU 9,013,201
Baichwal and Sugden, In: Gene Transfer, Kucherlapati (Ed), NY, Plenum Press, 117-148, 1986.
Banerji et al., Cell, 27(2 Pt 1):299-308, 1981.
Banerji et al., Cell, 33(3):729-740, 1983.
Barnes et al., J. Biol. Chem., 272(17):11510-7, 1997.
Benvenisty and Neshif, Proc. Natl. Acad. Sci. USA, 83:9551-9555, 1986.
Berkhout et al., Cell, 59:273-282, 1989.
Bhavsar et al., Genomics, 35(1):11-23, 1996.
Blanar et al., EMBO J., 8:1139, 1989.
Bodine and Ley, EMBO J., 6:2997, 1987.
Boshart et al., Cell, 41:521, 1985.
Bosher and Labouesse, Nat. Cell. Biol., 2:E31-E36, 2000.
Bosze et al., EMBO J., 5(7):1615-1623, 1986.
Braddock et al., Cell, 58:269, 1989.
Brinster et al., Proc. Natl. Acad. Sci. USA, 82(13):4438-4442, 1985.
Bristow, Cardiology, 92:3-6, 1999.
Bulla and Siddiqui, J. Virol., 62:1437, 1986.
Bush et al., Proc. Natl. Acad. Sci. U.S.A., 101:2870-2875, 2004.
Butler et al., Cancer Res., 60:5165-5170, 2000.
Butler et al., Clin. Cancer Res., 7:962-970, 2001.
Campbell and Villarreal, Mol. Cell. Biol., 8:1993, 1988.
Campbell et al., J. Mol. Biol., 180:1-19, 1984.
Campere and Tilghman, Genes and Dev., 3:537, 1989.
Campo et al., Nature, 303:77, 1983.
Capaldi et al., Biochem. Biophys. Res. Comm., 76:425-433, 1977.
Caplen et al., Gene, 252(1-2):95-105, 2000.
Carbonneau and Tonks, Annu. Rev. Cell. Biol., 8:463-93, 1992.
Celander and Haseltine, J. Virology, 61:269, 1987.
Celander et al., J. Virology, 62:1314, 1988.
Chandler et al., Cell, 33:489, 1983.
Chang et al., Hepatology, 14:124A, 1991.
Chang et al., Mol. Cell. Biol., 9:2153, 1989.
Chatterjee et al., Proc. Natl. Acad. Sci. USA, 86:9114, 1989.
Chen and Okayama, Mol. Cell Biol., 7:2745-2752, 1987.
Choi et al., Cell, 53:519, 1988.
Coffey et al., Cancer Res., 61:3591-3594, 2001.
Coffin, In: Virology, Fields et al. (Eds.), Raven Press, NY, 1437-1500, 1990.
Cohen et al., J. Cell. Physiol., 5:75, 1987.
Cook et al., Cell, 27:487-496, 1981.
Costa et al., Mol. Cell. Biol., 8:81, 1988.
Couch et al., Am. Rev. Resp. Dis., 88:394-403, 1963.
Coupar et al., Gene, 68:1-10, 1988.
Cripe et al., EMBO J., 6:3745, 1987.
Culotta and Hamer, Mol. Cell. Biol., 9:1376, 1989.
Dandolo et al., J. Virology, 47:55-64, 1983.
De Villiers et al., Nature, 312(5991):242-246, 1984.
Descharnps et al., Science, 230:1174-1177, 1985.
Dubensky et al., Proc. Natl. Acad. Sci. USA, 81:7529-7533, 1984.
Dunnrnon et al., J. Mol. Cell. Cardiol., 22:901-10, 1990.
Durand et al., Ann. Med., 27:311-317, 1995.
Edbrooke et al., Mol. Cell. Biol., 9:1908, 1989.
Edlund et al., Science, 230:912-916, 1985.
Egan and Weinberg, Nature, 365:781-783, 1993.
Eichhorn and Bristow, Circulation, 94:2285-2296, 1996.
Elbashir et al., Nature, 411(6836):494-498, 2001.
EP 0273085
EP 1,123,111
EP 1,170,008
EP 1,173,562
EP 1,174,438
EP 1,208,086
EP 1,233,958
Fechheimer et al., Proc Natl. Acad. Sci. USA, 84:8463-8467, 1987.
Feng and Holland, Nature, 334:6178, 1988.
Ferkol et al., FASEB J., 7:1081-1091, 1993.
Firak and Subramanian, Mol. Cell. Biol., 6:3667, 1986.
Fire et al., Nature, 391:806-811, 1998.
Foecking and Hofstetter, Gene, 45(1):101-105, 1986.
Forster and Symons, Cell, 49:211-220, 1987.
Fraley et al., Proc Natl. Acad. Sci. USA, 76:3348-3352, 1979
Franz et al., Cardoscience, 5(4):235-43, 1994.
Friedmann, Science, 244:1275-1281, 1989.
Fujita et al., Cell, 49:357, 1987.
Furumai et al., Cancer Res., 62:4916-21, 2002.
Gao et al., J. Biol. Chem., 277:25748-55, 2002.
Gao, et al., J. Biol. Chem., 15:8675-81, 1996.
Gefter, et al., Somatic Cell Genet., 3:231-236, 1977.
Gerlach et al., Nature (London), 328:802-805, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh-Choudhury et al., EMBO J., 6:1733-1739, 1987.
Gilles et al., Cell, 33:717, 1983.
Gloss et al., EMBO J., 6:3735, 1987.
Godbout et al., Mol. Cell. Biol., 8:1169, 1988.
Goding, In: Monoclonal Antibodies: Principles and Practice, 2d ed., Orlando, Fla., Academic Press, 60-61, 65-66, 71-74, 1986.
Gomez-Foix et al., J. Biol. Chem., 267:25129-25134, 1992.
Goodbourn and Maniatis, Proc. Natl. Acad. Sci. USA, 85:1447, 1988.
Goodbourn et al., Cell, 45:601, 1986.
Gopal, Mol. Cell Biol., 5:1188-1190, 1985.
Gopal-Srivastava et al., J. Mol. Cell. Biol., 15(12):7081-90, 1995.
Gottlicher et al., EMBO J., 20:6969-78, 2001.
Graham and Prevec, In: Methods in Molecular Biology: Gene Transfer and Expression Protocol, Murray (Ed.), Humana Press, Clifton, N.J., 7:109-128, 1991.
Graham and van der Eb, Virology, 52:456-467, 1973.
Graham et al., J. Gen. Virol., 36:59-72, 1977.
Greene et al., Immunology Today, 10:272, 1989
Grishok et al., Science, 287:2494-2497, 2000.
Grosschedl and Baltimore, Cell, 41:885, 1985.
Grozinger and Schreiber, Proc. Nat'l. Acad. Sci. U.S.A., 97:7835-40, 2000.
Grozinger et al., Proc. Natl. Acad. Sci. USA, 96:4868-4873, 1999.
Grunhaus and Horwitz, Seminar in Virology, 3:237-252, 1992.
Gschwendt et al., FEBS Lett., 392:77-80, 1996.
Han et al., Cancer Research, 60:6068-6074, 2000.
Hardie and Hanks (1995) The Protein Kinase Facts Books, Vol 1:7-20 Academic Press, San Diego, Calif.
Haribabu et al., EMBO J, 14:3679-86, 1995.
Harland and Weintraub, J. Cell Biol., 101:1094-1099, 1985.
Harlow and Lane, Antibodies: A Laboratory manual, Cold Spring Harbor Laboratory, 1988.
Haslinger and Karin, Proc. Natl. Acad. Sci. USA, 82:8572, 1985.
Hauber and Cullen, J. Virology, 62:673, 1988.
Haworth et al., J. Mol. Cardiol., 32:1013-1023, 2000.
Hayashi et al., Biochim. Biophys. Acta, 1450:99-106, 1999.
Hen et al., Nature, 321:249, 1986.
Hensel et al., Lymphokine Res., 8:347, 1989.
Hermonat and Muzycska, Proc. Nat'l Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Nat'l. Acad. Sci. USA 90:2812-2816, 1993.
Hinnebusch et al., J. Nutr., 132:1012-7, 2002.
Hirochika et al., J. Virol., 61:2599, 1987.
Hirsch et al., Mol. Cell. Biol., 10:1959, 1990.
Hoffmann et al., Bioconjugate Chem., 12:51-55, 2001.
Holbrook et al., Virology, 157:211, 1987.
Horlick and Benfield, Mol. Cell. Biol., 9:2396, 1989.
Horwich et al., J. Virol., 64:642-650, 1990.
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Isselbacher et al., In: Harrison's Principles of Internal Medicine, McGraw-Hill, NY, 416-431, 1994.
Jakobovits et al., Mol. Cell. Biol., 8:2555, 1988.
Jameel and Siddiqui, Mol. Cell. Biol., 6:710, 1986.
Japanese App. No. 2001/348340.
Jaynes et al., Mol. Cell. Biol., 8:62, 1988.
Johannes et al., J. Biol. Chem., 269(8):6140-8, 1994.
Johnson et al., Mol. Cell. Biol., 9:3393, 1989.
Jones and Shenk, Cell, 13:181-188, 1978.
Joyce, Nature, 338:217-244, 1989.
Jung et al., J. Med. Chem., 42:4669-4679, 1999.
Jung et al., Med. Chem. Lett., 7:1655-1658, 1997.
Jung, Curr. Med. Chem., 8:1505-11, 2001.
Kadesch and Berg, Mol. Cell. Biol., 6:2593, 1986.
Kaneda et al., Science, 243:375-378, 1989.
Kao et al., Genes Dev., 14:55-66, 2000.
Karin et al., Mol. Cell. Biol., 7:606, 1987.
Karin et al., Mol. Cell. Biol., 7:606, 1987.
Karlsson et al., EMBO J., 5:2377-2385, 1986.
Katinka et al., Cell, 20:393, 1980.
Kato et al., J. Biol. Chem., 266(6):3361-3364, 1991.
Katoh et al., J. Biol. Chem., 273:1511-18, 1998.
Kawamoto et al., Mol. Cell. Biol., 8:267, 1988.
Kelly et al., J. Cell Biol., 129(2):383-96, 1995.
Ketting et al., Cell, 99:133-141, 1999.
Kiledjian et al., Mol. Cell. Biol., 8:145, 1988.
Kim and Cook, Proc. Natl. Acad. Sci. USA, 84:8788-8792, 1987.
Kim et al., J. Am. Chem. Soc., 121:2056, 1999.
Kimura et al., Dev. Growth Differ., 39(3):257-65, 1997.
Kitazomo et al., J Clinical Endocr. Metabol., 86(7):3430-3435, 2001.
Klamut et al., Mol. Cell. Biol., 10:193, 1990.
Klaassen, In: The Pharmacological Basis of Therapeutics, Goodman and Gilman, Eds., Pergamon Press, 8th Ed., 49-61, 1990.
Klein et al., Nature, 327:70-73, 1987.
Koch et al., Mol. Cell. Biol., 9:303, 1989.
Kohler and Milstein, Eur. J. Immunol., 6:511-519, 1976.
Kohler and Milstein, Nature, 256:495-497, 1975.
Komastsu et al., Cancer Res., 61:4459-4466, 2001.
Kramer et al., Trends in Endoc. Metabolism, 12)₇):294-300, 2001.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
LaPointe et al., J. Biol. Chem., 263(19):9075-8, 1988.
Larsen et al., Proc. Natl. Acad. Sci. USA., 83:8283, 1986.
Laspia et al., Cell, 59:283, 1989.
Latimer et al., Mol. Cell. Biol., 10:760, 1990.
Le Gal La Salle et al., Science, 259:988-990, 1993.
Lee et al., Nature, 294:228, 1981.
Lee et al., Nucleic Acids Res., 12:4191-206, 1984.
Levinson et al., Nature, 295:79, 1982.
Levrero et al., Gene, 101:195-202, 1991.
Li et al., J. Biol. Chem., 271:19402-8, 1996.
Lin and Avery, Nature, 402:128-129, 1999.
Lin et al., Mol. Cell. Biol., 10:850, 1990.
Linnenbach et al., Proc Natl. Acad. Sci. USA, 85(1):74-8, 1988.
Lu et al., Proc. Natl. Acad. Sci. USA, 97:4070-4075, 2000.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Sarnow, Nature, 353:90-94, 1991.
Mai et al., J. Med. Chem., 45:1778-1784, 2002.
Majors and Varmus, Proc. Natl. Acad. Sci. USA, 80:5866, 1983.
Mann et al., Cell, 33:153-159, 1983.
Markowitz et al., J. Virol., 62:1120-1124, 1988.
Marks et al., Clin. Cancer Res., 7:759-760, 2001.
Marks et al., J. Natl. Cancer Inst., 92(15):1210-1216, 2000.
Massa et al., J. Med. Chem., 44:2069-2072, 2001.
McKinsey et al., Nature, 408:106-111, 2000b.
McKinsey et al., Proc. Natl. Acad. Sci. USA, 97:14400-14405, 2000a.
McKinsey et al., Trends Biochem. Sci., 27:40-47, 2002.
McNeall et al., Gene, 76:81, 1989.
Michel and Westhof, J. Mol. Biol., 216:585-610, 1990.
Miksicek et al., Cell, 46:203, 1986.
Montgomery et al., Proc. Natl. Acad. Sci. USA, 95:155-2-15507, 1998.
Mordacq and Linzer, Genes and Dev., 3:760, 1989.
Moreau et al., Nucl. Acids Res., 9:6047, 1981.
Moss et al., J. Gen. Physiol., 108(6):473-84, 1996.
Muesing et al., Cell, 48:691, 1987.
Ng et al., Nuc. Acids Res., 17:601, 1989.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Nishikawa et al., J. Biol. Chem., 276:3310-3318, 1997.
Ondek et al., EMBO J., 6:1017, 1987.
Ornitz et al., Mol. Cell. Biol., 7:3466, 1987.
Palmiter et al., Cell, 29:701, 1982.
Palmiter et al., Nature, 300:611, 1982.
Paskind et al., Virology, 67:242-248, 1975.
Pech et al., Mol. Cell. Biol., 9:396, 1989.
Pelletier and Sonenberg, Nature, 334:320-325, 1988.
Perales et al., Proc. Natl. Acad. Sci. USA, 91(9):4086-4090, 1994.
Perez-Stable and Constantini, Mol. Cell. Biol., 10:1116, 1990.
Physicians Desk Reference.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Racher et al., Biotech. Techniques, 9:169-174, 1995.
Ragot et al., Nature, 361:647-650, 1993.
Redondo et al., Science, 247:1225, 1990.
Reinhold-Hurek and Shub, Nature, 357:173-176, 1992.
Reisman and Rotter, Mol. Cell. Biol., 9:3571, 1989.
Remington's Pharmaceutical Sciences, 15^{th} ed., 1035-1038 and 1570-1580, Mack Publishing Company, PA, 1980.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Ridgeway, In: Vectors: A survey of molecular cloning vectors and their uses, Stoneham: Butterworth, 467-492, 1988.
Ripe et al., Mol. Cell. Biol., 9:2224, 1989.
Rippe et al., Mol. Cell Biol., 10:689-695, 1990.
Rittling et al., Nuc. Acids Res., 17:1619, 1989.
Rosen et al., Cell, 41:813, 1988.
Rosenfeld et al., Cell, 68:143-155,1992.
Rosenfeld et al., Science, 252:431-434,1991.
Roux et al., Proc. Natl. Acad. Sci. USA, 86:9079-9083, 1989.
Sakai et al., Genes and Dev., 2:1144, 1988.
Sambrook et al., Molecular Cloning: A Laboratory Manual, 2nd Ed., Cold Spring Harbor Press, Cold Spring Harbor, N.Y., 2001.
Sarver et al., Science, 247:1222-1225, 1990.
Satake et al., J. Virology, 62:970, 1988.
Saunders et al., Cancer Res., 59-399-409, 1999.
Scanlon et al., Proc. Natl. Acad. Sci. USA, 88:10591-10595, 1991.
Schaffner et al., J. Mol. Biol., 201:81, 1988.
Searle et al., Mol. Cell. Biol., 5:1480, 1985.
Sharp and Marciniak, Cell, 59:229, 1989.
Sharp and Zamore, Science, 287:2431-2433, 2000.
Sharp, Genes Dev., 13:139-141, 1999.
Shaul and Ben-Levy, EMBO J., 6:1913, 1987.
Sherman et al., Mol. Cell. Biol., 9:50, 1989.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Storz and Toker, Embo J., 22:109-20, 2003.
Stratford-Perricaudet and Perricaudet, In: Human Gene Transfer, Cohen-Haguenauer and Boiron (Eds.), John Libbey Eurotext, France, 51-61, 1991.
Stratford-Perricaudet et al., Hum. Gene. Ther., 1:241-256, 1990.
Stuart et al., Nature, 317:828, 1985.
Sturany et al., J. Biol. Chem., 276:3310-3318, 2001.
Su et al., Cancer Res., 60:3137-3142, 2000.
Sullivan and Peterlin, Mol. Cell. Biol., 7:3315, 1987.
Swartzendruber and Lehman, J. Cell. Physiology, 85:179, 1975.
Tabara et al., Cell, 99:123-132, 1999.
Takahashi et al., Antibiotics, 49:453, 1996.
Takebe et al., Mol. Cell. Biol., 8:466, 1988.
Taunton et al., Science, 272:371, 1996.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
The Merck Index, 11th Edition.
Thiesen et al., J. Virology, 62:614, 1988.
Tong et al., Nucleic Acids Res., 30:1114-23, 2002.
Top et al., J. Infect. Dis., 124:155-160, 1971.
Treisman, Cell, 42:889, 1985.
Tronche et al., Mol. Biol. Med., 7:173, 1990.
Trudel and Constantini, Genes and Dev. 6:954, 1987.
Tur-Kaspa et al., Mol. Cell Biol., 6:716-718, 1986.
Tyndell et al., Nuc. Acids. Res., 9:6231, 1981.
Valverde et al., Proc. Natl. Acad. Sci. USA, 91(18):8572-6, 1994.
Van den Wyngaert et al., FEBS Lett., 478:77-83, 2000.
Van Lint et al., Trends in Cell. Biol., 12:193-200, 2002.
Vannice and Levinson, J. Virology, 62:1305, 1988.
Varmus et al., Cell, 25:23-36, 1981.
Vasseur et al., Proc Natl. Acad. Sci. U.S.A., 77:1068, 1980.
Vigushin et al., Anticancer Drugs, 13:1-13, 2002.
Vigushin et al., Cancer Res., 5(Suppl), 1999.
Wagner et al., Proc. Natl. Acad. Sci. USA, 87(9):3410-3414, 1990.
Wang and Calame, Cell, 47:241, 1986.
Weber et al., Cell, 36:983, 1984.
Weinberger et al. Mol. Cell. Biol., 8:988, 1984.
Wincott et al., Nucleic Acids Res., 23(14):2677-2684, 1995.
Winoto and Baltimore, Cell, 59:649, 1989.
WO 00/44914
WO 01/14581
WO 01/18045
WO 01/36646
WO 01/38322
WO 01/42437
WO 01/68836
WO 01/70675
WO 02/26696
WO 02/26703
WO 02/30879
WO 02/46129
WO 02/46144
WO 02/50285
WO 02/51842
WO 84/03564
WO 98/33791
WO 99/32619
Wong et al., Gene, 10:87-94, 1980.
Workman and Kingston, Annu. Rev. Biochem., 67:545-579, 1998.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Yamano et al., Amer. Soc. Gene Ther., 2000.
Yamauchi-Takihara et al., Proc. Natl. Acad. Sci. USA, 86(10):3504-3508, 1989.
Yang et al., Proc. Natl. Acad. Sci. USA, 87:9568-9572, 1990.
Young et al., In: Handbook of Applied Therapeutics, 7.1-7.12 and 9.1-9.10, 1989.
Yutzey et al. Mol. Cell. Biol., 9:1397, 1989.
Zelenin et al., FEBS Lett., 280:94-96, 1991.
Zhang et al., Cell, 110:479-488, 2002.
Zhou et al., Proc. Natl. Acad. Sci. USA, 98:10572-10577, 2001.
Ziober and Kramer, J. Bio. Chem., 271(37):22915-22922, 1996.
Zugaza et al., Embo J., 15:6220-30, 1996.
Accesson No. NM002742.

1. A method of treating pathologic cardiac hypertrophy or heart failure comprising:
(a) identifying a patient having cardiac hypertrophy or heart failure; and
(b) administering to said patient an inhibitor of Protein Kinase D (PKD)

2. The method of claim 1, wherein said inhibitor of PKD is selected from the group consisting of resveratrol, indolocarbazoles, Godecke 6976 (Gö6976), staurosporine, K252a, Substance P (SP) analogues including [d-Arg(1),d-Trp(5,7,9), Leu(11)]SP, PKC inhibitor 109203X (GF-1), PKC inhibitor Ro 31-8220, GO 7874, Genistein, the specific Src inhibitors PP-1 and PP-2, chelerythrine, rottlerin, a PKD RNAi molecule, a PKD antisense molecule, a PKD ribozyme molecule or a PKD-binding single-chain antibody, or expression construct that encodes a PKD-binding single-chain antibody.

3. The method of claim 1, wherein administering the inhibitor of PKD is performed intravenously or by direct injection into cardiac tissue.

4. The method of claim 1, wherein administering comprises oral, transdermal, sustained release, controlled release, delayed release, suppository, or sublingual administration.

5. The method of claim 1, further comprising administering to said patient a second cardiac hypertrophic therapy.

6. The method of claim 5, wherein said second therapy is selected from the group consisting of a beta blocker, an ionotrope, a diuretic, ACE-I, AII antagonist, BNP, a Ca⁺⁺-blocker, or an HDAC inhibitor.

7. The method of claim 5, wherein said second therapy is administered at the same time as said inhibitor of PKD.

8. The method of claim 5, wherein said second therapy is administered either before or after said inhibitor of PKD.

9. The method of claim 1, wherein treating comprises improving one or more symptoms of pathologic cardiac hypertrophy.

10. The method of claim 1, wherein treating comprises improving one or more symptoms of heart failure.

11. The method of claim 9, wherein said one or more improved symptoms comprises increased exercise capacity, increased cardiac ejection volume, decreased left ventricular end diastolic pressure, decreased pulmonary capillary wedge pressure, increased cardiac output, or cardiac index, lowered pulmonary artery pressures, decreased left ventricular end systolic and diastolic dimensions, decreased left and right ventricular wall stress, decreased wall tension, increased quality of life, and decreased disease related morbidity or mortality.

12. A method of preventing pathologic hypertrophy or heart failure comprising:
(a) identifying a patient at risk of developing pathologic cardiac hypertrophy or heart failure; and
(b) administering to said patient an inhibitor of PKD.

13. The method of claim 12, wherein said inhibitor of PKD is selected from the group consisting of resveratrol, indolocarbazoles, Godecke 6976 (Gö6976), staurosporine, K252a, Substance P (SP) analogues including [d-Arg(1),d-Trp(5,7,9), Leu(11)]SP, PKC inhibitor 109203X (GF-1), PKC inhibitor Ro 31-8220, PKC inhibitor GO 7874, Genistein, the specific Src inhibitors PP-1 and PP-2, chelerythrine, rottlerin, a PKD RNAi molecule, a PKD antisense molecule, a PKD ribozyme molecule or a PKD-binding single-chain antibody, or expression construct that encodes a PKD-binding single-chain antibody.

14. The method of claim 12, wherein administering the inhibitor of PKD is performed intravenously or by direct injection into cardiac tissue.

15. The method of claim 12, wherein administering comprises oral, transdermal, sustained release, controlled release, delayed release, suppository, or sublingual administration.

16. The method of claim 12, wherein the patient at risk may exhibit one or more of a list of risk factors comprising long standing uncontrolled hypertension, uncorrected valvular disease, chronic angina, recent myocardial infarction, congenital predisposition to heart disease or pathological hypertrophy.

17. The method of claim 12, wherein the patient at risk may be diagnosed as having a genetic predisposition to cardiac hypertrophy.

18. The method of claim 12, wherein the patient at risk may have a familial history of cardiac hypertrophy.

19. A method of assessing an inhibitor of PKD for efficacy in treating cardiac hypertrophy or heart failure comprising:
(a) providing an inhibitor of PKD;
(b) treating a cell with said inhibitor of PKD; and
(c) measuring the expression of one or more cardiac hypertrophy parameters,
wherein a change in said one or more cardiac hypertrophy parameters, as compared to one or more cardiac hypertrophy parameters in a cell not treated with said inhibitor of PKD, identifies said inhibitor of PKD as an inhibitor of cardiac hypertrophy or heart failure.

20. The method of claim 19, wherein said cell is a myocyte.

21. The method of claim 19, wherein said cell is an isolated myocyte.

22. The method of claim 21, wherein said myocyte is a cardiomyocyte

23. The method of claim 20, wherein said myocyte is comprised in isolated intact tissue.

24. The method of claim 20, wherein said myocyte is a neonatal rat ventricular myocyte.

25. The method of claim 19, wherein said cell is an H9C2 cell.

26. The method of claim 22, wherein said cardiomyocyte is located in vivo in a functioning intact heart muscle.

27. The method of claim 26, wherein said functioning intact heart muscle is subjected to a stimulus that triggers a hypertrophic response in one or more cardiac hypertrophy parameters.

28. The method of claim 27, wherein said stimulus is aortic banding, rapid cardiac pacing, induced myocardial infarction, or transgene exression.

29. The method of claim 27, wherein said stimulus is a chemical or pharmaceutical agent.

30. The method of claim 29, wherein said chemical or pharmaceutical agent comprises angiotensin II, isoproterenol, phenylepherine, endothelin-I, vasoconstrictors, antidiuretics.

31. The method of claim 27, wherein said one or more cardiac hypertrophy parameters comprises right ventricular ejection fraction, left ventricular ejection fraction, ventricular wall thickness, heart weight/body weight ratio, right or left ventricular weight/body weight ratio, or cardiac weight normalization measurement.

32. The method of claim 20, wherein said myocyte is subjected to a stimulus that triggers a hypertrophic response in said one or more cardiac hypertrophy parameters.

33. The method of claim 32, wherein said stimulus is expression of a transgene.

34. The method of claim 32, wherein said stimulus is treatment with a drug.

35. The method of claim 19, wherein said one or more cardiac hypertrophy parameters comprises the expression level of one or more target genes in said myocyte, wherein expression level of said one or more target genes is indicative of cardiac hypertrophy.

36. The method of claim 35, wherein said one or more target genes is selected from the group consisting of ANF, α-MyHC, β-MyHC, α-skeletal actin, SERCA, cytochrome oxidase subunit VIII, mouse T-complex protein, insulin growth factor binding protein, Tau-microtubule-associated protein, ubiquitin carboxyl-terminal hydrolase, Thy-1 cell-surface glycoprotein, or MyHC class I antigen.

37. The method of claim 35, wherein the expression level is measured using a reporter protein coding region operably linked to a target gene promoter.

38. The method of claim 37, wherein said reporter protein is luciferase, β-gal, or green fluorescent protein.

39. The method of claim 35, wherein the expression level is measured using hybridization of a nucleic acid probe to a target mRNA or amplified nucleic acid product.

40. The method of claim 19, wherein said one or more cardiac hypertrophy parameters comprises one or more aspects of cellular morphology.

41. The method of claim 40, wherein said one or more aspects of cellular morphology comprises sarcomere assembly, cell size, or cell contractility.

42. The method of claim 19, wherein said one or more cardiac hypertrophy parameters comprises total protein synthesis.

43. The method of claim 19, further comprising measuring cell toxicity.

44. The method of claim 19, wherein said cell expresses a mutant class II HDAC protein lacking one or more phosphorylation sites.

45. The method of claim 19, wherein said measuring comprises measuring the activity or expression of a gene selected from the group consisting of an atrial natriuretic factor gene, a β-myosin heavy chain gene, a cardiac actin gene and an α-skeletal actin gene.

46. The method of claim 19, wherein said measuring comprises measuring the phosphorlyation of class-II HDAC's.

47. The method of claim 19, wherein said measuring comprises measuring the nuclear export of class-II HDAC's.

48. The method of claim 19, wherein said measuring comprises measuring the association of class-II HDAC's and Mef-2.

49. The method of claim 48, wherein the measuring further comprises measuring for an enhancement of class-II HDAC association with Mef-2.

50. The method of claim 49, wherein said enhancement is measured by an increase in Mef-2 dependent transcription.

51. The method of claim 19, wherein said treating is performed in vitro.

52. The method of claim 19, wherein said treating is performed in vivo.

53. The method of claim 19, wherein said cell is part of a transgenic, non-human mammal.

54. A method of identifying an inhibitor of cardiac hypertrophy or heart failure comprising:
(a) providing a PKD;
(b) contacting the PKD with a candidate inhibitor substance; and
(c) measuring the kinase activity of said PKD,
wherein a decrease in the kinase activity of the PKD identifies said candidate inhibitor substance as an inhibitor of cardiac hypertrophy or heart failure.

55. The method of claim 54, where said PKD is purified away from whole cells.

56. The method of claim 55, wherein said cells are heart cells.

57. The method of claim 54, wherein said PKD is located in an intact cell.

58. The method of claim 57, wherein said intact cell is a myocyte.

59. The method of claim 58, wherein said myocyte is a cardiomyocyte.

60. The method of claim 54, wherein a decrease in kinase activity is measured as a decrease in phosphorylation of HDAC.

61. The method of claim 60, wherein HDAC is a class-II HDAC.

62. The method of claim 54, wherein the candidate inhibitor substance is an interfering RNA.

63. The method of claim 54, wherein the candidate inhibitor substance is an antibody preparation.

64. The method of claim 63, wherein the antibody preparation comprises single chain antibodies.

65. The method of claim 54, wherein the candidate inhibitor substance is an antisense construct.

66. The method of claim 54, wherein said inhibitor is an enzyme, chemical, pharmaceutical, or small compound.

67. The method of claim 54, wherein said inhibitor of PKD is selected from the group consisting of resveratrol, indolocarbazoles, Godecke 6976 (Gö6976), staurosporine, K252a, Substance P (SP) analogues including [d-Arg(1),d-Trp(5,7,9), Leu(11)]SP, PKC inhibitor 109203X (GF-1), PKC inhibitor Ro 31-8220, GO 7874, Genistein, the specific Src inhibitors PP-1 and PP-2, chelerythrine, rottlerin.

68. The method of claim 54, wherein said inhibitor blocks binding of PKD to class II HDAC's.

69. The method of claim 68, wherein the method of the blockage of binding is measured by co-immunoprecipitation.

70. The method of claim 54, wherein said inhibitor blocks PKD phosphorylation of class II HDAC's.

71. The method of claim 54, wherein said inhibitor enhances HDAC association with Mef-2 or other class II HDAC regulated transcription factors.

72. A transgenic, non-human mammal, the cells of which comprise a heterologous PKD gene under the control of a promoter active in eukaryotic cells.

73. The transgenic mammal of claim 72, wherein said mammal is a mouse.

74. The transgenic mammal of claim 72, wherein said heterologous PKD gene is human.

75. The transgenic mammal of claim 72, wherein said promoter is a tissue specific promoter.

76. The transgenic mammal of claim 75, wherein the tissue specific promoter is a muscle specific promoter.

77. The transgenic mammal of claim 75, wherein the tissue specific promoter is a heart muscle specific promoter.

78. The transgenic mammal of claim 75, wherein the muscle specific promoter is selected from the group consisting of myosin light chain-2 promoter, alpha actin promoter, troponin 1 promoter, Na⁺/Ca²⁺ exchanger promoter, dystrophin promoter, creatine kinase promoter, alpha7 integrin promoter, brain natriuretic peptide promoter, myosoin heavy chain promoter, ANF promoter, and alpha B-crystallin/small heat shock protein promoter.

79. The transgenic mammal of claim 72, wherein said kinase is constitutively active.

80. The transgenic mammal of claim 72, wherein said kinase is a dominant negative.

81. A transgenic, non-human mammal, the cells of which comprise a PKD gene under the control of a heterologous promoter active in the cells of said non-human mammal.

82. The transgenic mammal of claim 81, wherein said mammal is a mouse.

83. The transgenic mammal of claim 81, wherein said PKD gene is human.

84. The transgenic mammal of claim 83, wherein said promoter is active in eukaryotic cells.

85. The transgenic mammal of claim 84, wherein said promoter is a tissue specific promoter.

86. The transgenic mammal of claim 85, wherein the tissue specific promoter is a muscle specific promoter.

87. The transgenic mammal of claim 85, wherein the tissue specific promoter is a heart muscle specific promoter.

88. A transgenic, non-human mammal, the cells of which lack one or both native PKD alleles.

89. The mammal of claim 88, wherein one or more genes have been knocked out by homologous recombination.

90-99. (canceled)

100. The method of claim 1, wherein treating pathologic cardiac hypertrophy or heart failure further comprises increasing exercise tolerance, reducing hospitalizations, improving quality of life, decreasing morbidity, or decreasing mortality in a subject with heart failure or cardiac hypertrophy.
